(19) United States
(12) Patent Application Publication
(10) Pub. No.: US 2024/0336761 A1
(43) Pub. Date: Oct. 10, 2024

(54) Method and Reactor System For Depolymerizing A Polymer Using A Reusable Catalyst

(71) Applicant: Ioniqa Technologies B.V., Eindhoven (NL)

(72) Inventors: Egor Vasilyevich FUFACHEV, Eindhoven (NL); Joost Robert WOLTERS, Eindhoven (NL); Michael Josef DE GROOT, Eindhoven (NL)

(73) Assignee: Ioniqa Technologies B.V., Eindhoven (NL)

(21) Appl. No.: 18/291,764

(22) PCT Filed: Jul. 26, 2022
(86) PCT No.: PCT/NL2022/050440

§ 371 Date: Jan. 24, 2024

(30) Foreign Application Priority Data
Jul. 29, 2021 (NL) 2028883

(51) Int. Cl. C08J 11/24; B01D 17/02; B01D 21/26; B01D 29/56; B01D 36/04; B01J 19/00; B01J 19/24; C07C 67/475

(52) U.S. Cl. CPC C08J 11/24; B01D 17/0214; B01D 17/0217; B01D 21/262; B01D 29/56; B01D 36/045; B01J 19/0013; B01J 19/245; C07C 67/475; B01J 2219/0004; B01J 2219/00054; C08J 2367/02

(57) ABSTRACT

A method and reactor system for depolymerizing a polymer is described. The method comprises the steps of providing the polymer and a solvent in a reactor to obtain a reaction mixture, the solvent being capable of reacting with the polymer to degrade the polymer into at least repeating units; providing a reusable catalyst in the reaction mixture being capable of catalyzing said degradation; degrading the polymer in the reaction mixture at degradation reaction conditions to obtain a depolymerized mixture comprising at least light oligomers having from 2 to 4 repeating units inclusively; removing unreacted polymer, solid particles and optionally very heavy oligomers from the depolymerized mixture after exiting the reactor; recovering at least a part of the reusable catalyst from the depolymerized mixture; and recovering the light oligomers from the depolymerized mixture. During recovery of the reusable catalyst, the depolymerized mixture comprises heavy oligomers having at least 5 repeating units.

### FIELD OF THE INVENTION

The invention relates to a method for depolymerizing a polymer using a reactive solvent for degrading the polymer and a reusable catalyst. The reusable catalyst catalyzes the degradation reaction of the polymer. In the method, a relatively large amount of the reusable catalyst applied may be recovered and put to use again. The invention also relates to a reactor system in which the invented method may be carried out.

### BACKGROUND ART

There is an increasing awareness that the large amount of polymers used nowadays for a variety of purposes should be recycled in order to prevent an increasing amount of polymer waste. Incineration is one possibility, however undesirable for obvious reasons. Mechanical shredding and milling of used polymers may be another solution to the problem of accumulating polymer waste. However, the recycled polymer properties are then degraded and they frequently end up as filler for other materials. Circular recycling (chemical recycling) is clearly the solution of choice. In circular recycling, the polymer to be recycled is depolymerized into its repeating units, such as the monomer units from which the polymer is made. Depolymerization methods may also yield light oligomers, i.e. oligomers having 2 to 4 repeating units inclusively (dimers, trimers and tetramers). The monomers, and optionally the light oligomers, resulting from the degradation reaction may be used again in making a new polymer.

Degradation of used polymers may be hindered by the fact that such polymers are typically present in a product that may contain a plurality of materials, and suitable separation methods should be provided for separating the polymer to be recycled from the product. As a consequence, a significant amount of used polymers is still used as a fuel, which is burned. Polymers may comprise fillers and other additives to enhance their properties. They may be natural or man-made. They may also be combined with another polymer in a polymer blend, or form a copolymer. Combining polymers with other materials such as metals, glass and even stone is also possible. All this makes recycling more difficult.

Further, chemical recycling of polymers such as polyethylene terephthalate (PET) for instance is considered to be cost-efficient only when performed at a relatively high scale using high capacity recycling lines of at least 50 ktons/year for instance. Such recycling lines may then be combined with polymer production sites of polymer producers, which tend to be of large scale as well.

A further difficulty seems to be assuring a consistent and continuous waste sourcing, such as bottles and textiles for instance, in a required extensive amount at one single site. Yet a further issue may be that, even when the problem of separation of the desired polymer to be recycled from a product has been solved satisfactorily, the process of degradation of the polymer into monomers and optionally light oligomers has proven to be difficult. Many known methods are not selective enough, or are deficient in terms of a too low conversion. An efficient conversion of the polymers to the desired products (monomers and/or light oligomers) is desirable, at the same time minimizing production of waste in terms of side products. In other words, a relatively high yield (selectivity times conversion) is a desirable goal in depolymerization methods.

The depolymerization reaction itself may be carried out in a variety of reactor types, such as batch and continuous reactors. The latter make use of flow chemistry, wherein a chemical depolymerization reaction is proceeding in a continuously flowing medium contrary to what happens in batch production.

Catalysts are often used for synthesis of polymers, but less for degradation of waste polymers. A reason may be that catalytic activity may be sensitive to contaminants that are typically present in waste polymers. Catalysts may function only properly under relatively pure and clean conditions. As a result hereof, catalysts that are used in degradation processes may need to be replaced regularly.

To catalyze the degradation reaction and increase yield, use is typically made of catalysts, either heterogeneous or homogeneous. When using a heterogeneous catalyst, selectivity and conversion may be less than with homogeneous catalysts, and the amount of available catalysts to choose from is rather limited. Homogeneous catalysts however tend to be less recoverable. This may lead to contamination of the reaction products, which is less desirable.

A catalyst is typically provided in a reactive solvent that is capable of reacting with the polymer to degrade the polymer into its monomers and light oligomers. Since catalysts may be quite expensive, it would be desirable to be able to recover a relatively large amount of catalyst after having degraded a polymer in the reaction mixture comprising the polymer, the reactive solvent and the catalyst. Such recovered catalyst could then be reused a second time and preferably many more times.

Specialized catalysts for catalyzing depolymerization reactions do exist, such as the one described in WO 2016/105200 A1 and in US 2018/0371206 A1. The catalyst disclosed therein relates to a catalyst complex comprising three distinguishable elements: a nanoparticle, a bridging moiety attached to the nanoparticle, for instance by but not limited to a covalent bond, and a catalyst entity attached to the bridging moiety, for instance by, but not limited to, a covalent bond. The catalyst has been shown to be highly selective and to produce relatively high yield in depolymerization. The nanoparticle is preferably of a magnetic nature or may be magnetized to a sufficient extend under relatively modest magnetic fields. The use of magnetic nanoparticles improves recoverability of the catalyst, for instance by magnetic attraction after use.

A method for depolymerizing a polymer, in particular a terephthalate polymer, is also disclosed in WO 2021/089803 A1. The disclosed method comprises supplying waste material comprising the polymer and a solvent into a reactor vessel and heating the waste material to depolymerize the polymer into monomer, dimer, trimer and/or oligomer. A catalyst complex may be added to catalyze the depolymerization. The method forms a first part and a second part in the reactor vessel, wherein the second part comprises heavier agglomerates, for instance including polyolefins, that were present in the waste material. The liquid first part comprising monomer, dimer, trimer and/or oligomer is fed through a heat exchanger and then to a separator. The second part is also fed to the separator in which it phase-separates from the first part. A centrifuge provided downstream from the separator then separates the first phase from the second phase, and catalyst may be recovered from the second phase that includes polymer.

Although the above exemplified catalyst is recoverable to a satisfactory extend, being able to recover a substantial amount of other catalysts and an increased amount of the exemplified catalyst while retaining the catalyzing activity of the recovered catalyst remains an important goal. A small waste of catalyst could be acceptable, for instance in the order of a few percent or less, such as less than 5 wt. %, more preferably less than 4 wt. %, even more preferably less than 2 wt. %, and even more preferably less than 1 wt. % of the catalyst. A substantially complete recovery of the catalyst however is most preferred.

Therefore, there is a need for a method and reactor system for depolymerizing a polymer, wherein a substantial amount of the catalyst used in the depolymerization reaction may be recovered and reused.

### SUMMARY OF THE INVENTION

Thereto, according to the invention is provided a method for depolymerizing a polymer, the method comprising the steps of:
a) providing the polymer and a solvent in a reactor to obtain a reaction mixture, the solvent being capable of reacting with the polymer to degrade the polymer into its monomers and oligomers;
b) providing a reusable catalyst in the reaction mixture being capable of catalyzing said degradation;
c) degrading the polymer in the reaction mixture at degradation reaction conditions to obtain a depolymerized mixture comprising monomers and at least light oligomers having from 2 to 4 repeating units; and removing unreacted polymer, solid particles and optionally very heavy oligomers having more than 200 repeating units from the depolymerized mixture after exiting the reactor;
d) recovering at least a part of the reusable catalyst from the depolymerized mixture;
e) recovering the monomers and optionally the light oligomers from the depolymerized mixture;
wherein, during recovery of the reusable catalyst in step d), the depolymerized mixture further comprises heavy oligomers having at least 5 repeating units and at most 200 repeating units.

In another aspect of the invention, there is provided a reactor system for recycling of waste material comprising a polymer suitable for depolymerization. The reactor system comprises:
a first reactor vessel with at least one inlet for waste material and another inlet for providing a reusable catalyst to the first reactor vessel being capable of catalyzing the depolymerization reaction of the polymer, and an outlet, which first reactor vessel is configured for depolymerizing the polymer into its monomers and oligomers; and which outlet is configured for exiting a depolymerized mixture;
a first filter unit arranged downstream of the outlet and configured for removing unreacted polymer, solid particles and very heavy oligomers having more than 200 repeating units from the depolymerized mixture after exiting the first reactor vessel, such that at least monomers and light oligomers having from 2 to 4 repeating units and heavy oligomers having at least 5 and at most 200 repeating units remain present in the depolymerized mixture;
a heat exchanger provided downstream of the outlet and the first filter unit;
a separating unit provided downstream of the heat exchanger and preferably comprising at least one centrifuge, the separating unit being configured for recovering at least a part of the reusable catalyst from the depolymerized mixture and/or for recovering the monomers and the light oligomers having from 2 to 4 repeating units from the depolymerized mixture;
a conduit system connecting the reactor system components, as well as pressure means for circulation purposes through the conduit system, wherein the conduit system comprises a feedback conduit for feeding the recovered part of the reusable catalyst back into the first reactor vessel.

The reactor system is particularly suitable for carrying out the invented method.

### DETAILED DESCRIPTION OF THE INVENTION

According to the invention, a method for depolymerizing a polymer is provided, the method comprising the steps of:
a) providing the polymer and a solvent in a reactor to obtain a reaction mixture, the solvent being capable of reacting with the polymer to degrade the polymer into its monomers and oligomers;
b) providing a reusable catalyst in the reaction mixture being capable of catalyzing said degradation;
c) degrading the polymer in the reaction mixture at degradation reaction conditions to obtain a depolymerized mixture comprising monomers and at least light oligomers having from 2 to 4 repeating units: removing unreacted polymer, solid particles and very heavy oligomers having more than 200 repeating units from the depolymerized mixture after exiting the reactor;
d) recovering at least a part of the reusable catalyst from the depolymerized mixture;
e) recovering the monomers and the light oligomers from the depolymerized mixture;
wherein, during recovery of the reusable catalyst in step d), the depolymerized mixture further comprises heavy oligomers having at least 5 repeating units and at most 200 repeating units.

The inventors have found out that, although the presence of polymers during the step of recovering the reusable catalyst from the depolymerized mixture, as disclosed by WO 2021/089803 A1, may improve the catalyst recovery, providing heavy oligomers only with at most 200 repeating unit in the depolymerized mixture in step d) surprisingly improves catalyst recovery to a major extent, as demonstrated by the experimental section of the present disclosure. In other words, the molecular weight of the heavy oligomers in the depolymerized mixture during step d) should not be too high since recovery of the catalyst then deteriorates. Keeping the molecular weight of the heavy oligomers in the depolymerized mixture during the recovery step d) between limits, as claimed, i.e. having at least 5 repeating units and at most 200 repeating units, allows an improved and even a substantially complete reuse of the catalyst which may significantly reduce the cost of the depolymerization, and further increase its efficiency. The catalyst may be homogeneous or heterogeneous, the latter being preferred.

In the context of the present application, monomers are defined as molecules that contain one repeating unit of the polymer, whereas oligomers comprise molecules that contain at least 2 repeating units. The light oligomers relate to molecules that contain from 2 to 4 repeating units inclusively, such as dimers, trimers and tetramers, whereas the heavy oligomers are defined as having at least 5 repeating units. Since the heavy oligomers are still oligomers, their average molecular weight cannot be too high. It has been established that a too high molecular weight of the heavy oligomers has a decreasing effect on catalyst recovery. It appears therefore that an optimum molecular weight range of the heavy oligomers is operative. In a preferred embodiment therefore, an upper bound of the amount of repeating units in the heavy oligomers is 200, more preferably 100, even more preferably 50, even more preferably 40, even more preferably 30, and most preferably 20. Very heavy oligomers are defined as oligomers having more repeating units than the heavy oligomers, i.e. having an amount of repeating units that is above the upper bound defined above. It should also be mentioned that the heavy oligomers will show a molecular weight distribution. Therefore, defining the heavy oligomers as having from 5-200 repeating units does not necessarily mean that they should contain molecules having 5 repeating units, or 200 repeating units. They do in this embodiment however comprise molecules having lengths that fall within the range of 5-200 repeating units. For instance, in the case of depolymerized PET, the molecular weight of a 5-mer (with 2 carboxylic end groups) is 934 g/mol and the molecular weight of a 200-mer (with 2 glycol end groups) is 38462 g/mol.

The diagram below illustrates the depolymerization of PET for instance using ethylene glycol as reactive solvent. In such case the heavy oligomers are represented by the oligomers with m≥5, and preferably m≤200.

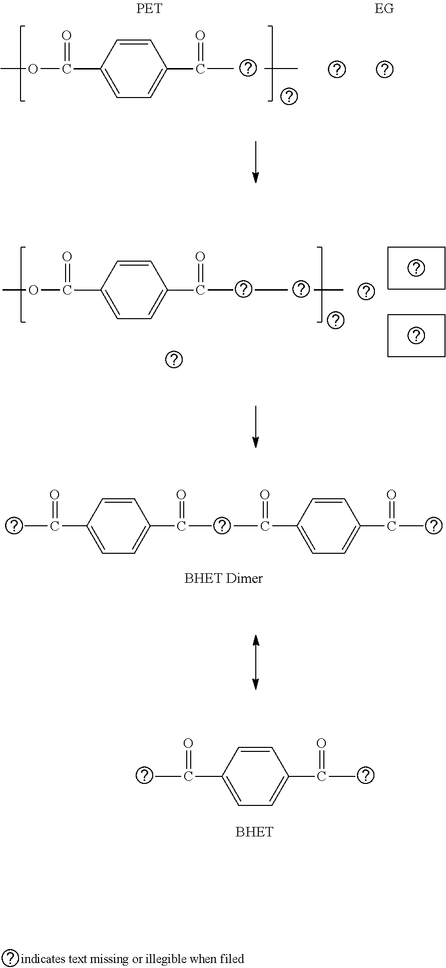

The depolymerized mixture is the mixture that remains after the degrading step c). Depending on reaction conditions and according to embodiments of the invention, the depolymerized mixture may contain the monomers, the light oligomers, and the heavy oligomers, containing more than 5 repeating units. It may even contain unreacted polymer and/or the very heavy oligomers. Other material present in the waste polymer feedstock may also be present in the depolymerized mixture when exiting the reactor vessel. Such other material may for instance comprise polyolefins, possibly other radical-polymerized polymers such as PVC and polystyrene as well as metal, for instance aluminum, glass and stone particles and the like. Also, other condensation polymers than the one to be depolymerized are examples of such other material. The primary polymer to be depolymerized is polyethylene terephthalate (PET) in a preferred embodiment. However, the invention is in principle not limited to the depolymerization of PET, and the method may be used for depolymerizing other polymers and polyesters as well.

The heavy oligomers present in the depolymerized mixture during recovery of the reusable catalyst in step d), may originate from different sources. They may for instance originate from the depolymerized mixture itself, or from another depolymerized polymer, or they may be provided as such. In one embodiment, a method is provided wherein said heavy oligomers comprise repeating units of said polymer to be degraded. In other words, the heavy oligomers are those that derive from the polymer to be degraded. They may be provided separately or may originate from the depolymerized mixture. In other embodiments, the heavy oligomers may originate from another polymer, or may have been synthetized and added to the depolymerized mixture in step d), i.e. the step of recovering at least a part of the reusable catalyst from the depolymerized mixture. It is also possible to combine different sources of the heavy oligomers.

In a suitable embodiment, a method is provided wherein said monomers and oligomers are formed by degrading the polymer in the reaction mixture at the degradation reaction conditions during step c). In order to promote the formation of the heavy oligomers and prevent that these heavy oligomers are further degraded to the light oligomers, a method according to another embodiment is provided wherein the degradation reaction in step c) is stopped prematurely by removing the degradation reaction conditions.

The degradation reaction conditions may be removed in a number of ways. In one embodiment, a method is provided wherein the degradation reaction temperature is reduced relative to the degradation reaction temperature needed for full degradation of said polymer into its monomers and light oligomers. In another embodiment, a method is provided wherein the degradation reaction time is reduced relative to the degradation reaction time needed for full degradation of said polymer into its monomers and light oligomers, including, dimers, trimers and/or tetramers. In yet another embodiment, a method is provided wherein the degradation reaction temperature and time are reduced relative to the degradation reaction temperature and time needed for full degradation of said polymer into its monomers and light oligomers.

In a suitable method according to an embodiment, the degradation reaction time is at most 0.95 times, more preferably at most 0.8, and even more preferably at most 0.5 times the degradation reaction time needed for full degradation of said polymer into its monomers and light oligomers.

The heavy oligomers need not to originate from the polymer to be degraded. For instance a method according to an embodiment may be provided wherein said heavy oligomers also comprise other heavy oligomers having at least 5 repeating units, and preferably at most 200 repeating units, of another polymer that differs from said polymer to be degraded. It is also possible that the polymer to be degraded is a block copolymer and that certain blocks after degradation of the copolymer form the heavy oligomers.

The other polymer may for instance comprise a condensation polymer that is degradable by said solvent at said degradation reaction conditions, and that has been added to the reaction mixture. It may also be that the other heavy oligomers originate from the other polymer after degradation of said other polymer in a separate process. The heavy oligomers may also be synthesized in a separate oligomerization process. In such embodiments, the heavy oligomers are then available through a separate source.

According to yet another embodiment of the invention, a method is provided wherein said heavy oligomers (originating from the polymer to be degraded or from another source) are added to the depolymerized mixture after step c) and before or during step d). This embodiment provides for further flexibility. It is also possible that the heavy oligomers originate from the degradation reaction of the polymer to be degraded and further from another source.

In yet another preferred embodiment, a method is provided wherein the degrading step c) comprises the steps of:
c1) degrading said polymer of step a) substantially fully to form the monomers and/or the light oligomers having from 2 to 4 repeating units;
c2) adding an additional amount of said polymer to the reaction mixture after step c1); and
c3) degrading said additional amount of said polymer such that the depolymerized mixture contains said heavy oligomers derived from said additional amount of said polymer.

The amount of the heavy oligomers in the depolymerized mixture is not particularly critical. It should not be too high since this would decrease the yield of the monomers and the light oligomers in the depolymerized mixture. It should not be too low in order to negatively affect the amount of recovered catalyst.

A preferred embodiment provides a method as claimed wherein the amount of said heavy oligomers in the catalyst recovery step d) ranges from 0.1-50 wt. %, preferably from 1-30 wt. %, and more preferably from 5-25 wt. % relative to the total weight of the monomers and light oligomers in the depolymerized mixture.

According to another embodiment of the method, the amount of monomer in the depolymerized mixture ranges from 5-95 wt. %, preferably from 10-85 wt. %, and more preferably from 15-75 wt. % relative to the total weight of the monomers, the light oligomers, and the heavy oligomers in the depolymerized mixture.

For the degradation, the solid polymer is provided in a reactive solvent for the monomer. As such, the method may be viewed as a solid-liquid degradation process supported by addition of a recoverable catalyst. As reactive solvent, alcohols may be used for instance. The alcohol is preferably chosen from the group of aliphatic alcohols. Suitably, use is made of an alcohol with a boiling point above 150° C., which is further able to participate in the decomposition of the polymer as a reagent. In this perspective, polyols are preferred. Suitable polyols are for instance glycerol, propylene glycol and ethylene glycol. A most preferred alcohol is ethylene glycol. This results in glycolysis of the polymer, more particularly a condensation polymer, for instance a polyester. Due to the presence of two alcohol-groups per molecule, the monomer is provided with one free alcohol-group, and is therewith capable of reacting again with an acid to form an ester. Such free alcohol-group would not be available, when use is made of monoalcohols, such as methanol.

However, the claimed method is not limited to the use of glycolysis, and also works with depolymerization methods that are based on hydrolysis and/or methanolysis, or on other depolymerization principles. It is not excluded to use a mixture of alcohols. A mixture of an alcohol and another polar organic solvent, such as a ketone or aldehyde is not excluded. However, it is preferred that merely one alcohol would participate as a reagent in the decomposition, so as to prevent formation of different monomers.

Suitably, the polymer to be degraded is a condensation polymer, for instance chosen from polyesters, polyethers, polycarbonates, polyimides and polyamides. Representative examples include PET (polyethylene terephthalate), PEF (polyethylene furanoate), PTT (polytrimethylene terephthalate), PBT (polybutylene terephthalate), PLA (polylactic acid).

More generically, the polymer may be selected from natural polymers, biobased polymers, biodegradable polymers, polymers formed (directly or indirectly) from fossil fuels, and combinations thereof. In an example the polymer is at least one of a polyester, a polyether, such as poly-oxymethylene (POM), polyethylene glycol (PEG), polypropylene glycol (PPG), polytetramethyleneglycol (PTMG), polyethylene oxide (PEO), polypropylene oxide (PPO), polytetrahydrofuran (PTHF), and polytetramethyleneetherglycol (PTMEG), a polypeptide, a polyamide, a polyamine, a polycondensate, preferably a polyester, such as poly carboxylic ester, wherein the poly carboxylic ester is preferably selected from polyethylene terephthalate (PET), polyethylene furanoate (PEF), polybutylene terephthalate (PBT), polytrimethylene terephthalate (PTT), polyglycolic acid (PGA), polylactic acid (PLA), polycaprolactone (PCL), polyethylene adipate (PEA), polyhydroxyalkanoate (PHA), polyhydroxy butyrate (PHB), polyethylene naphthalate (PEN), Poly (3-hydroxy butyrate-co-3-hydroxyvalerate) (PHBV), and a polycondensate of 4-hydroxy benzoic acid and 6-hydroxynaphthalene-2-carboxylic acid (VECTRAN). In other words, a heavy variety of polymers may be degraded by the present method. Some adjustments may be necessary, e.g. in terms of catalyst used, temperature applied, solvent used, etc. The present method is best suited for degradation using glycolysis, such as in degradation of polyesters and polyethers, in particular PET and PEF. Suitably, the polymer material to be degraded is a waste polymer material, for instance obtained from bottles or textiles. This waste material may comprise one or more additives, such as colorants.

The invention is in particular useful for the depolymerization of terephthalate polymers. Terephthalate polymers are a group of polyesters comprising terephthalate in the backbone. The most common example of a terephthalate polymer is polyethylene terephthalate, also known as PET. Alternative examples include polybutylene terephthalate, polypropylene terephthalate, polyethylene isophthalate, poly pentaerythrityl terephthalate and copolymers thereof, such as copolymers of ethylene terephthalate and polyglycols, for instance polyoxyethylene glycol and poly (tetramethylene glycol) copolymers. PET is one of the most common polymers and it is highly desired to recycle PET by depolymerization thereof into reusable raw material.

One preferred way of depolymerization is glycolysis, which is catalysed. Typically, as a result of the use of an alcohol such as ethylene glycol, a reaction mixture comprising monomer bis (2-hydroxyethyl) terephthalate (BHET) and its light oligomers may be formed. The amount of light oligomers formed may depend on a number of process parameters, such as the polymer:solvent ratio, i.e. the PET:EG ratio in case of depolymerizing PET with EG as reactive solvent. According to the degradation process, the terephthalate polymer is depolymerized by glycolysis in the presence of a catalyst. At the end of the depolymerization process, water may be added and a phase separation may occur. This enables to separate a first phase comprising the BHET monomer unit and light oligomers from a second phase comprising catalyst, the heavy oligomers and, optionally other additives such as colorants and the like. The first phase may comprise impurities in dissolved form and as dispersed particles. The BHET monomers may be recovered by means of crystallization for instance.

A high purity of the monomers and the light oligomers is required in order to reuse them for polymerization. As is well-known, any contaminant may have an impact on the subsequent polymerization reaction from the raw materials. Moreover, since terephthalate polymers are used for food and also medical applications, strict rules apply to prevent health issues.

A preferred method for the depolymerization of the, preferably condensation, polymer, such as PET, includes several steps, typically comprising the provision of the catalyst, the polymer and the reactive solvent, such as an alcohol, heating to an elevated temperature so as to achieve an acceptable reaction rate. The solid polymer disperses into the reactive solvent (said dispersing typically also comprising at least some depolymerization), and the polymer is decomposed into oligomers and monomers. Then, a separation of a solution with the dissolved monomers and any dissolved light oligomers on the one hand and less- or non-dissolved parts on the other hand is carried out. The less- or non-dissolved parts typically comprise the catalyst, and, as it has turned out the heavy oligomers as well. Further non-dissolved parts may include any pigment, dyes or other colorants and fillers present in the solid polymer.

The depolymerization method may be carried out batch-wise, continuous, semi-continuous, and according to combinations thereof. A continuous configuration is preferred.

The temperature and pressure of the method may be selected according to the polymer to be degraded and the catalyst used. Typically, a somewhat higher temperature is preferred in terms of reaction velocity, while a lower temperature is preferred in terms of energy consumption. Likewise a higher pressure is somewhat preferred, while in view of reducing complexity of the reactor system a lower pressure may be preferred, for instance a pressure of about 100 kPa.

The elevated temperature used in depolymerization may be chosen dependent on the polymer and the catalyst by routine experimentation and is typically chosen at most 20° C. lower than the boiling point of the reactive solvent used. In the case of depolymerization of polyesters such as PET, ethylene glycol is the preferred reactive solvent, and the elevated temperature is suitably selected in the range of 150-250° C., more preferably in the range of 180-220° C., and most preferably in the range of 190-210° C. In order to stop the degradation reaction, the temperature may be reduced to below 160° C. for instance, or lower. Typical reaction times range from 30 sec to 24 hr and more, depending on the choice of other conditions. In the method, the dispersing of the solid polymer and decomposition of the polymer into oligomers and monomers may occur simultaneously.

It is a further advantage of the present method that it is relatively insensitive (e.g. in terms of yield) to the use of mixed polymers. Mixed polymers may for instance relate to a combination of two or more different types of polymers, such as different polyesters, such as PET, polyethylene furanoate (PEF), polytrimethylene terephthalate (PTT), and polybutylene terephthalate (PBT), or to a combination of one type of polymer having different properties, such as color, origin, and so on, and combinations thereof. The method is also relatively insensitive to contaminants, comprising additives present in the polymer to be degraded, such as pigments, fillers, and the like.

In a preferred embodiment, a method is provided, comprising a further step of removing unreacted polymer and optionally very heavy oligomers with more repeating units than the heavy oligomers from the depolymerized mixture after step c) and before step d). Removing said materials from the depolymerized mixture may significantly increase the amount of catalyst that is recovered.

In order to use said removed material to advantage a method according to a useful embodiment is provided wherein the removed unreacted polymer and optional very heavy oligomers are depolymerized to a substantially full conversion into monomers and light oligomers in a separate second reactor vessel by applying the steps a) to c) of claim 1. The fully depolymerized mixture that results from the depolymerization in the second reactor vessel may then preferably be introduced in the depolymerized mixture before step d).

It has also been found beneficial for the recovery yield of the catalyst to provide a method according to yet another embodiment wherein other material present in the depolymerized mixture, such as polyolefins for instance, is at least partly, and preferably completely, removed from the depolymerized mixture after step c) and before step d).

Several methods exist for recovering the catalyst from the depolymerized mixture. In case the catalyst comprises magnetic particles for instance, the catalyst may be removed by subjecting the depolymerized mixture to a magnetic field. An improved embodiment however relates to a method wherein the catalyst recovery step d) comprises a phase forming step, comprising forming a first phase primarily containing the monomers and light oligomers, and a second phase primarily containing said heavy oligomers and the catalyst, wherein the phase forming step comprises cooling the depolymerized mixture down from the reaction temperature, preferably to a temperature below 160° C. In this process, said heavy oligomers may be first dissolved in the reaction mixture during the degrading step c), and may during the phase forming step at least partly precipitate from the depolymerized mixture. It turns out that the heavy oligomers having an upper bound of the amount of repeating units of 200, more preferably of 100, even more preferably of 50, even more preferably of 40, even more preferably of 30, and most preferably of 20 perform optimally in that an increased amount of catalyst may be recovered.

It may be possible to add water to the depolymerized mixture in the phase forming step to create a hydrophilic solution as a first phase that substantially contains the monomers and the light oligomers, and a second phase, which substantially contains the heavy oligomers and the catalyst.

However, a preferred embodiment relates to a method wherein the phase forming step is carried out substantially without adding water to the depolymerized mixture.

The inventors have found out that the precipitated heavy oligomers surprisingly comprise a substantial amount of the catalyst. The current embodiment allows to remove the heavy oligomers together with the catalyst from the depolymerized mixture, for instance by centrifugation. After degradation the obtained mixture may be cooled down, preferably to a temperature of 50-150° C., more preferably 80-110° C. At this temperature a separation is performed, most particularly by centrifugation. To further increase the recovered catalyst yield, separation may be performed in a plurality of centrifuges provided in series. Advantageously, disc stack centrifuges may also be used. In a disc stack centrifuge, a separating means is rotated at high speed creating high G-forces.

The step of providing the reusable catalyst in an embodiment comprises reusing the recovered catalyst, preferably by adding the recovered catalyst from said second phase to the reaction mixture.

In accordance with a preferred embodiment of the invention, the method further comprises separating said first phase primarily containing the monomer and light oligomers from said second phase primarily containing said heavy oligomers and the catalyst, wherein said separating is carried out at a temperature below 100° C., preferably by centrifugation.

In another embodiment of the method, the monomer is obtained in a crystallization step wherein the monomer is crystallized from said first phase primarily containing the monomer and the light oligomers, after the separating step. The formed crystals (for instance BHET crystals) are then removed from the mother liquor by filtration for instance. Any reactive solvent remaining after the claimed method, typically a mixture of an alcohol and optionally water, may be recovered and recycled after reducing the optional water content to about zero water content.

The invention may be carried out using any catalyst suitable for the purpose. Suitable catalysts are reusable and recoverable, and preferably comprise heterogeneous catalysts. In a depolymerization method according to an embodiment, the catalyst then forms a dispersion in the reaction mixture during step c).

Several of the possible heterogeneous depolymerization catalysts are based on ferromagnetic and/or ferrimagnetic materials. Also anti-ferromagnetic materials, synthetic magnetic materials, paramagnetic materials, superparamagnetic materials, such as materials comprising at least one of Fe, Co, Ni, Gd, Dy, Mn, Nd, Sm, and preferably at least one of O, B, C, N, such as iron oxide, such as ferrite, such as magnetite, hematite, and maghemite can be used. The catalyst particles may comprise nanoparticles.

The catalyst particles catalyze the depolymerization reaction. In this depolymerization reaction individual molecules of the polymer are released via a catalytic reaction out of the solid polymer, which polymer is for instance semi-crystalline. This release results in dispersing of polymer material into the reactive solvent and/or dissolving of individual polymer molecules in the reactive solvent. Such dispersing and/or dissolving is believed to further enhance depolymerization from polymer into monomers and oligomers.

One class of suitable catalysts includes the transition metals, in their metallic or ionic form. The ionic form includes free ions in solutions and in ionic bonds or covalent bonds. Ionic bonds form when one atom gives up one or more electrons to another atom. Covalent bonds form with interatomic linkage that result from the sharing of an electron pair between two atoms. The transition metal may be chosen from the first series of transition metals, also known as the 3d orbital transition metals. More particularly, the transition metal is chosen from iron, nickel and cobalt. Since cobalt however is not healthy and iron and nickel particles may be formed in pure form, iron and nickel particles are most preferred. Furthermore, use can be made of alloys of the individual transition metals.

If a catalytic particle is made of metal it may be provided with an oxide surface, which may further enhance catalysis. The oxide surface may be formed by itself, in contact with air, in contact with water, or the oxide surface may be applied deliberately.

Most preferred is the use of iron particles. Besides that iron particles are magnetic, they have been found to catalyze the depolymerization of PET for instance to conversion rates into monomer of 70-90% within an acceptable reaction time of at most 6 hours, however depending on catalyst loading and other processing factors such as the PET/solvent ratio.

Non-porous metal particles, in particular transition metal particles, may be suitably prepared by thermal decomposition of carbonyl complexes such as iron pentacarbonyl and nickel tetracarbonyl. Alternatively, iron oxides and nickel oxides may be prepared via exposure of the metals to oxygen at higher temperatures, such as 400° C. and above. A non-porous particle may be more suitable than a porous particle, since its exposure to the alcohol may be less, and therefore, the corrosion of the particle may be less as well, and the particle may be reused more often for catalysis. Furthermore, due to the limited surface area, any oxidation at the surface may result in a lower quantity of metal-ions and therewith a lower level of ions that are present in the product stream as a leached contaminant to be removed therefrom.

Another class of suitable catalysts includes particles based on earth alkali elements selected from beryllium (Be), magnesium (Mg), calcium (Ca), strontium (Sr) and barium (Ba), and their oxides. A preferred earth alkali metal oxide is magnesium oxide (MgO). Other suitable metals include but are not limited to titanium (Ti), zirconium (Zr), manganese (Mn), zinc (Zn), aluminum (Al), germanium (Ge) and antimony (Sb), as well as their oxides, and further alloys thereof. Also suitable are precious metals, such as palladium (Pd) and platinum (Pt). MgO and ZnO have been found to catalyze the depolymerization of PET for instance to conversion rates into monomer of 70-90% within an acceptable reaction time, again depending on catalyst loading and other processing factors such as the PET/solvent ratio. Suitable catalysts based on hydrotalcites are also considered.

Preferably, the catalytic particles are selected so as to be substantially insoluble in the (alcoholic) reactive solvents, also at higher temperatures of more than 100° C. One oxide that readily tends to dissolve at higher temperatures in an alcohol such as glycol, is for instance, such as amorphous SiO₂, are less suited.

The preferred concentration of catalyst is 1 wt % relative to the amount of PET or less. Good results have also been achieved with a catalyst loading below 0.2 wt % and even below 0.1 wt % relative to the amount of PET. Such a low loading of the catalyst is highly beneficial, and the invented method allows to recovering an increased amount of the nanoparticle catalyst.

Non-porous particles according to the invention have a surface area suitably less than 10 m²/g, more preferably at most 5 m²/g, even more preferably at most 1 m²/g. In another embodiment, the surface area is at least 3 m²/g. The porosity is suitably less than 10⁻² cm³/g or for instance at most 10⁻³ cm³/g. Porous particles may also be used, generally exhibiting a larger surface area.

Nanoparticles may also be used as or in a depolymerization catalyst. Such nanoparticles have a small diameter and a surface area of in the range of 10-1000 m²/g, more preferably of 50-500 m²/g, and most preferably from 80-150 m²/g, or more. Significant adsorption of the condensation polymer with these kind of nanoparticles takes place, which is believed to result in faster depolymerization and therewith an economically feasible process. To separate such nanoparticles a number of options are available.

The catalyst nanoparticle is preferably of a magnetic nature, either comprising a magnetic material, or having the ability to be magnetized sufficiently under relatively modest magnetic fields, such as being applied in the present method. Suitably, the magnetic nanoparticles contain an iron, nickel and/or cobalt, in their oxidic or metallic form, or combinations thereof. Iron oxide, for instance but not exclusively in the form of Fe₃O₄ is preferred. Another suitable example is α- and/or γ-Fe₂O₃.

From the alloys a suitable example is CoFe₂O₄. Other preferred examples are NiFe₂O₄, Ni₂Fe₂O₅ or NiO.

It has been found that the nanoparticles should be 17enzotrently small for the catalyst complex to function as a catalyst, therewith degrading the polymer into smaller units, wherein the yield of these smaller units, and specifically the monomers thereof, is high enough for commercial reasons. It has further been found that the nanoparticles should be sufficiently large in order to be able to reuse by recovering the present catalyst. It is economically unfavorable that the catalyst would be removed with either waste or degradation product obtained. Suitable nanoparticles have an average diameter in the range of from 2 up to 500 nm, more preferably in the range of from 3 up to 200 nm, even more preferably from 4 up to 100 nm. It has been found that e.g. in terms of yield and recovery of catalyst complex a rather small size of particles of 5-50 nm is optimal. It is noted that the term “size” relates to an average diameter of the particles, wherein an actual diameter of a particle may vary somewhat due to characteristics thereof. In addition aggregates may be formed e.g. in the solution. These aggregates typically have sizes in a range of 50-200 nm, such as 80-150 nm, e.g. around 100 nm. It is preferred to use nanoparticles comprising iron oxide.

Particle sizes and a distribution thereof can be measured by light scattering, for instance using a Malvern Dynamic light Scattering apparatus, such as a NS500 series. In a more laborious way, typically applied for smaller particle sizes and equally well applicable to large sizes representative electron microscopy pictures are taken and the sizes of individual particles are measured on the picture. For an average particle size, a number average may be taken. In an approximation the average may be taken as the size with the highest number of particles or as a median size.

A preferred heterogeneous catalyst that may be used in the invention is a catalyst complex comprising catalyst particles, such as those disclosed above, and a catalyst entity that is associated with or attached to the catalyst particles via a linking group, wherein the catalyst entity comprises a cationic moiety having a positive charge and a negative moiety having a negative charge. The catalyst particles are preferably nanoparticles, and the catalyst particles may in themselves also exhibit some catalytic activity.

The catalyst complex (ABC) comprises three distinguishable elements: a (nano) particle (A), a linking group (B) attached to the particle chemically, such as by a covalent bond, or physically, such as by adsorption, and a catalyst entity (C) that may be chemically, such as covalently, bonded to the linking group. The linking group preferably does not fully cover the nanoparticle surface, such as in a core-shell particle.

The particles of this catalyst complex are preferably based on ferromagnetic and/or ferrimagnetic materials. Also anti-ferromagnetic materials, synthetic magnetic materials, paramagnetic materials, superparamagnetic materials, such as materials comprising at least one of Fe, Co, Ni, Gd, Dy, Mn, Nd, Sm, and preferably at least one of O, B, C, N, such as iron oxide, such as ferrite, such as hematite (Fe₂O₃), magnetite (Fe₃O₄), and maghemite (Fe₂O₃, γ-Fe₂O₃) may be used. In view of costs, even when fully or largely recovering the present catalyst complex, relatively cheap particles are preferred, such as particles comprising Fe. A further advantage of particles of iron or iron oxides is that they have highest saturation magnetisation, making it easier to separate the particles via a magnetic separator. And even more importantly, the iron oxide nanoparticles have a positive impact on the degradation reaction. The iron oxide may further contain additional elements such as cobalt and/or manganese, for instance CoFe₂O₄.

The catalyst particles that are used in the catalyst complex according to the invention may be coated at least partly with a protective coating. For example, Fe₃O₄-particles may be coated with a material to promote the formation of a stable suspension, and eventually protect the particles from oxidation to Fe₂O₃, which would have different magnetic properties. Thus, at least a part of the surface of the catalyst particles may be coated with materials such as polyethyleneimine (PEI), polyethylene glycol (PEG), silicon oil, fatty acids like oleic acid or stearic acid, silane, a mineral oil, an amino acid, or polyacrylic acid or, polyvinylpyrrolidone (PVP). Carbon is also possible as coating material. The coating may be removed before or during the catalytic reaction. Ways to remove the coating may for instance comprise using a solvent wash step separately before using it in the reactor, or by burning in air. Removal of the coating however is not essential.

The present catalyst entity comprises at least two moieties. A first moiety relates to a moiety having a positive charge (cation). A second moiety relates to a moiety, typically a salt complex moiety, having a negative charge (anion). The negative and positive charges typically balance one another. It has been found that the positively and negatively charged moieties have a synergistic and enhancing effect on the degradation process of waste terephthalate polymer in terms of conversion and selectivity.

The positively charged moiety (cation) may be aromatic or aliphatic, and/or heterocyclic. The cationic moiety may be aliphatic and is preferably selected from guanidinium (carbamimidoylazanium), ammonium, phosphonium and sulphonium. A non-aromatic or aromatic heterocyclic moiety preferably comprises a heterocycle, having at least one, preferably at least two hetero-atoms. The heterocycle may have 5 or 6 atoms, preferably 5 atoms. The positively charged moiety may be an aromatic moiety, which preferably stabilizes a positive charge. Typically the cationic moiety carries a positive charge on the hetero-atom. The hetero-atom may be nitrogen N, phosphor P or sulphur S for instance. Suitable aromatic heterocycles are pyrimidines, imidazoles, piperidines, pyrrolidine, pyridine, pyrazol, oxazol, triazol, thiazol, 19enzotriazo, 19enzotriazole, isoquinol and viologen-type compounds (having f.i. two coupled pyridine-ring structures). Particularly preferred is an imidazole structure, which results in an imidazolium ion. Suitable cationic moieties having N as hetero-atom comprise imidazolium, (5-membered ring with two N), piperidinium (6-membered ring with one N), pyrrolidinium (5-membered ring having one N), and pyridinium (6-membered ring with one N). Preferred imidazolium cationic moieties comprise butylmethylimidazolium (bmim⁺), ethylimidazolium, or butylimidazolium (bim⁺). Other suitable cationic moieties include but are not limited to triazolium (5-membered ring with 3 N), thiazolidium (5-membered ring with N and S), and (iso) quiloninium (two 6-membered rings (naphthalene) with N).

In a preferred method, the cationic moiety of the catalyst entity is selected from at least one of an imidazolium group, a piperidinium group, a pyridinium group, a pyrrolidinium group, a sulfonium group, an ammonium group, and a phosphonium group.

Said cationic moiety may have one ore more substituents, which one ore more substituents is preferably selected an alkyl moiety. In particular examples, said alkyl moiety has a length of C₁-C₆, such as C₂-C₄. In specific examples, said imidazolium group has two substituents R₁, R₂ attached to one of the two nitrogen atoms, respectively, said piperidinium group has two substituents R₁, R₂ attached to its nitrogen atom, said pyridinium group has two substituents R₁, R₂ wherein one of the two substituents R₁, R₂ is attached to its nitrogen atom, said pyrrolidinium group has two substituents R₁, R₂ attached to its nitrogen atom, said sulphonium group has three substituents R₁, R₂, R₃ attached to its sulphur atom, said ammonium group has four substituents R₁, R₂, R₃, R₄ attached to its nitrogen atom, and said phosphonium group has four substituents R₁, R₂, R₃, R₄ attached to its phosphor atom, respectively.

The negatively charged moiety (anion) may relate to an anionic complex, but alternatively to a simple ion, such as a halide. It may relate to a salt complex moiety, preferably a metal salt complex moiety, having a two- or three-plus charged metal ion, such as Fe³⁺, Al³⁺, Ca²⁺, Zn²⁺ and Cu²⁺, and negatively charged counter-ions, such as halogenides, e.g. Cl⁻, F⁻, and Br⁻. In an example the salt is a Fe³⁺ comprising salt complex moiety, such as an halogenide, e.g. FeCl₄⁻. Alternatively, use can be made of counter-ions without a metal salt complex, such as halides as known per se.

The linking group may comprise a bridging moiety for attaching the catalyst entity to the catalyst particle. The present catalyst entity and particle are combined by the bridging moiety by attaching the catalyst entity to the catalyst particle. The attachment typically involves a physical or chemical bonding between a combination of the bridging moiety and the catalyst entity on the one hand and the catalyst particle on the other hand. Particularly, a plurality of bridging moieties is attached or bonded to a surface area of the present catalyst particle. Suitable bridging moieties comprise a weak organic acid, silyl comprising groups, and silanol. More particularly, therefore, the bridging moiety comprises a functional group for bonding to the oxide of the particle and a second linking group for bonding to the catalyst entity. The functional group is for instance a carboxylic acid, an alcohol, a silicic acid group, or combinations thereof. Other acids such as organic sulphonic acids are not excluded. The linking group comprises for instance an end alkylene chain attached to the cationic moiety, with the alkylene chain typically between C₁ and C₆, for instance propylene and ethylene. The linking group may be attached to the cationic moieties such as the preferred imidazolium moiety. In the attached state, a BC complex then for instance comprises imidazolium having two alkylgroups, such as butylmethylimidazolium (bmim+) or ethylmethylimidazolium as an example.

The bridging moiety is suitably provided as a reactant, in which the linking group is functionalized for chemical reaction with the catalyst entity. For instance, a suitable functionalization of the linking group is the provision as a substituted alkyl halide. Suitable reactants for instance include 2-chloropropyltrialkoxysilane and 2-bromopropyltrialkoxysilane. The alkoxy-group is preferably ethoxy, although methoxy or propoxy groups are not excluded. It is preferred to use trialkoxysilanes, although dialkyldialkoxysilanes and trialkyl-monoalkoxysilanes are not excluded. In the latter cases, the alkyl groups are preferably lower alkyl, such as C₁-C₄ alkyl. At least one of the alkyl groups is then functionalized, for instance with a halide, as specified above.

The said reactant is then reacted with the catalyst entity. Preferably, this reaction generates the positive charge on the cationic moiety, more particularly on a hetero-atom in the, preferably heterocyclic, cationic moiety. The reaction is for instance a reaction of a (substituted) alkyl halide with a hetero-atom, such as nitrogen, containing cationic moiety, resulting in a bond between the hetero-atom and the alkyl-group. The hetero-atom is therewith charged positively, and the halide negatively. The negatively charged halide may thereafter be strengthened by addition of a Lewis acid to form a metal salt complex. One example is the conversion of chloride to FeCl₄⁻.

According to the present invention, the bridging moiety and the catalyst entity bonded thereto are provided in an amount of (mole bridging moiety/gr magnetic particle) 5*10⁻⁶-0.1, preferably 1*10⁻⁵-0.01, more preferably 2*10⁻⁵-10⁻³, such as 4*10⁻⁵-10⁻⁴. It is preferred to have a relatively large amount available in terms of an effective optional recovery of the catalyst complex, whereas, in terms of amount of catalyst and costs thereof, a somewhat smaller amount may be more preferred.

The catalyst complex is in preferred embodiments used in a weight ratio of the catalyst complex to the polymer ranging from 0.001:10 to 1.0:10, preferably 0.005:10 to 0.5:10.

Another aspect of the invention relates to a reactor system for recycling of waste material comprising a polymer suitable for depolymerization. The reactor system comprises:
a first reactor vessel with at least one inlet for waste material and another inlet for providing a reusable catalyst to the first reactor vessel being capable of catalysing the depolymerization reaction of the polymer, and an outlet, which first reactor vessel is configured for depolymerizing the polymer into its monomers and oligomers; and which outlet is configured for exiting a depolymerized mixture;
a first filter unit arranged downstream of the outlet and configured for removing unreacted polymer, solid particles and optionally very heavy oligomers from the depolymerized mixture after exiting the first reactor vessel, such that at least light oligomers having from 2 to 4 repeating units inclusively, and heavy monomers having at least 5 and optionally at most 200 repeating units remain present in the depolymerized mixture;
a heat exchanger provided downstream of the outlet and the first filter unit;
a separating unit provided downstream of the heat exchanger and preferably comprising at least one centrifuge, the separating unit being configured for recovering at least a part of the reusable catalyst from the depolymerized mixture and/or for recovering the monomers and the light oligomers having from 2 to 4 repeating units inclusively from the depolymerized mixture;—a conduit system connecting the reactor system components, as well as pressure means for circulation purposes through the conduit system, wherein the conduit system comprises a feedback conduit for feeding the recovered part of the reusable catalyst back into the first reactor vessel.

The reactor system is particularly suitable for carrying out the invented method.

An improved embodiment of the reactor system is characterized in that the first filter unit is configured for removing very heavy oligomers with more than 200 repeating units from the depolymerized mixture after exiting the first reactor vessel, more preferably configured for removing very heavy oligomers with more than 100, even more preferably more than 50, even more preferably more than 40, even more preferably more than 30, and most preferably more than 20 repeating units from the depolymerized mixture after exiting the first reactor vessel. Suitable filter units may comprise a strainer or other unit capable of performing the filtering function.

One skilled in the art will be able to design the first filter unit to let through the heavy oligomers and withheld unreacted polymer, solid particles and the very heavy oligomers having more than 200 repeating units. The same accounts for the optional second and third filter units described further below. A suitable method to select retention capacity of the filter unit (for instance its mesh size) would be to force the depolymerized mixture though a filter unit and collect the retentate and filtrate. By measuring the (number or weight) average molecular weight of the retentate and the filter unit allows to correlate the retention capacity of the filter unit with the desired average molecular weight of the retentate and/or the filtrate. Any method for determining the average molecular weight may be used and suitable methods comprise Gel Permeation Chromatography (GPC) and Matrix Assisted Laser Desorption Ionization Time of Flight Mass Spectrometry (MALDITOF MS). It has been found that suitable mesh size may range from 20 μm-1000 μm, more preferably from 50 μm-800 μm, even more preferably from 100 μm-600 μm, and most preferably from 150 μm-500 μm.

In another embodiment, the reactor system further comprises a separate second reactor vessel provided to receive the removed unreacted polymer and optional very heavy oligomers from the first filter unit, wherein the second reactor is configured for depolymerizing the removed unreacted polymer and optional very heavy oligomers to a substantially full conversion into monomers and light oligomers. Preferably, the second reactor vessel has an outlet configured for exiting the fully depolymerized mixture, and a conduit connecting the outlet with the heat exchanger. The conduit connecting the outlet with the heat exchanger preferably comprises a third filter unit arranged downstream of the outlet and configured for removing solid particles from the fully polymerized mixture.

Another useful embodiment of the invention provides a reactor system that further comprises a second filter unit configured for removing other material present in the depolymerized mixture, such as polyolefins for instance, at least partly and preferably completely from the depolymerized mixture, wherein the second filter unit is provided downstream of the heat exchanger and upstream from the separating unit.

Yet another embodiment provides a reactor system that further comprises a source of heavy oligomers other than the first reaction vessel, the source having an outlet configured for exiting the heavy oligomers from the source, and a conduit connected to the outlet and configured for adding the heavy oligomers to the depolymerized mixture in or downstream of the heat exchanger and/or in or upstream from the separating unit. This embodiment provides further flexibility in adjusting the desired amount of heavy oligomers in the depolymerized mixture for improved catalyst recovery.

The separating unit provided downstream of the heat exchanger preferably comprises at least one centrifuge. The separating unit is configured for recovering at least a part of the reusable catalyst from the depolymerized mixture and/or for recovering the light oligomers having from 2 to 4 repeating units inclusively from the depolymerized mixture. According to the invention, the separating unit may be configured for substantially completely recovering the reusable catalyst from the depolymerized mixture. A further improved reactor system according to an embodiment, is characterized in that the separating unit comprises a plurality of centrifuges provided in series, wherein any centrifuge may more preferably comprise a disc stack centrifuge.

Another practical embodiment provides a reactor system wherein at least one solvent buffer vessel is arranged upstream of the first and/or second reactor vessel, an inlet of the at least one solvent buffer vessel being connected to the feedback conduit, and an outlet thereof being connected to the first reactor vessel and/or to the second reactor vessel.

### BRIEF DESCRIPTION OF THE FIGURES

These and other aspects of the method and the reactor system of the invention will be further elucidated with reference to the figures, which are purely diagrammatical in nature and not drawn to scale. In the figures:

FIG. 1 shows a reactor system according to an embodiment of the invention;

FIG. 2 shows a reactor system according to another embodiment of the invention;

FIG. 3 shows a photograph of a polyethylene terephthalate (PET) waste material used;

FIG. 4 shows a graph of the concentration of iron (Fe) in the BHET and the mother liquor, obtained after depolymerization of the material of FIG. 3 using a Fe-based catalyst, and after separation of the BHET for a number of embodiments according to the invention and for a number of Comparative Examples;

FIG. 5 shows a graph of the separation efficiency of the Fe-based catalyst for a number of embodiments according to the invention and for a number of Comparative Examples;

FIG. 6 shows a photograph of another polyethylene terephthalate (PET) waste material used;

FIG. 7 shows a graph of the concentration of iron (Fe) in the BHET and the mother liquor, obtained after depolymerization of the material of FIG. 6 using a Fe-based catalyst, and after separation of the BHET, for a number of embodiments according to the invention and for a number of Comparative Examples;

FIG. 8 shows a graph of the separation efficiency of the Fe-based catalyst for a number of embodiments according to the invention and for a number of Comparative Examples;

FIG. 9 shows a photograph of heavy oligomers before (picture a) and after (picture b) separation of the catalyst of the material used in Example 5;

FIG. 10 shows a graph of the concentration of Mg in the BHET and the mother liquor, obtained after depolymerization of the material of FIG. 6 using a Mg-based catalyst, and after separation of the BHET for a number of embodiments according to the invention and for a number of Comparative Examples;

FIG. 11 shows a graph of the separation efficiency of the Mg-based catalyst for a number of embodiments according to the invention and for a number of Comparative Examples;

FIG. 12 shows a photograph of yet another polyethylene terephthalate (PET) waste material used;

FIG. 13 shows a graph of the concentration of iron (Fe) in the BHET and the mother liquor after depolymerization of the material of FIG. 12 using a Fe-based catalyst and after separation of the BHET for an embodiment according to the invention and a Comparative Example; and

FIG. 14 finally shows a graph of the separation efficiency of the Fe-based catalyst for an embodiment according to the invention, and a Comparative Example.

### DETAILED DESCRIPTION OF ILLUSTRATED EMBODIMENTS

In the following, equal or corresponding parts in different figures will be referred to with equal reference numerals. The illustrated embodiments are intended for explanation and illustration and are not intended to limit the scope of the claims.

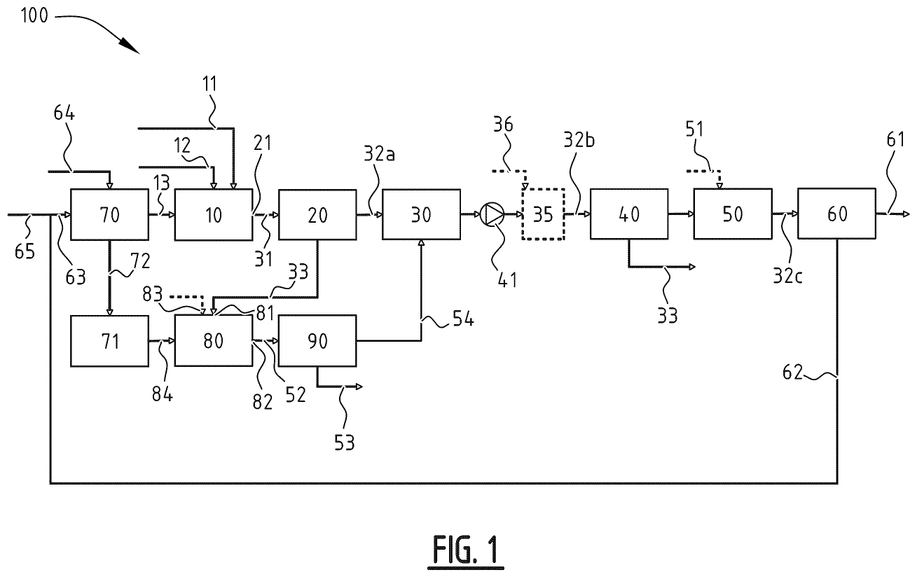

FIG. 1 shows the reactor system 100 according to a first embodiment, which comprises a reactor vessel 10, provided with a first inlet 11, a second inlet 12 and a third inlet 13, as well as an outlet 21. The reactor vessel 10 is configured for depolymerization of polymers, in particular condensation polymers, and the depolymerized mixture exits the vessel 10 through the outlet 21. Other material present in the feedstock may also be separated from the outlet 21. Such other material for instance comprises polyolefins, possibly other radical-polymerized polymers such as PVC and polystyrene as well as metal, such as aluminum, glass and stone. Also, other condensation polymers than the one to be depolymerized are examples of such other material. The primary condensation polymer to be depolymerized is polyethylene terephthalate (PET) in a preferred embodiment. However, the invention is in principle not limited to the depolymerization of PET, and the reactor system may be used for depolymerizing other polymers and polyesters as well.

The reactor system 100 shown in FIG. 1 is configured as a continuous system such that the reactor vessel 10 is continuously provided with polymer waste material, solvent and catalyst to sustain the depolymerization reaction. In such continuous system, it may be desirable to make use of a cascade of reactors. The polymer material, solvent and catalyst constitute a reaction mixture, of which the composition changes in the course of the depolymerization. According to an embodiment of the invention, the conditions in the first reactor vessel 10 are such that the primary polymer (the polymer to be degraded) added to it is only partly depolymerized, i.e. is depolymerized to a non-full conversion. Thus, only a part of the primary polymer, such as PET, is depolymerized into light oligomers and monomers, while another part is depolymerized into heavy oligomers. It may even be that some undissolved polymer exits the first reactor vessel 10. The waste material loaded into the reactor vessel 10 via the first inlet 11 is typically in the form of flakes. The solvent added to the vessel 10 through the second inlet 12 is preferably an alcohol, more preferably ethylene glycol. The catalyst may be any catalyst suitable for the purpose and may for instance be based on an ionic-liquid functionalized magnetic nanoparticle or aggregate thereof. Preferred magnetic nanoparticles are iron oxide particles and cobalt-iron oxide particles. The presence of other metals in addition to iron and/or cobalt is not excluded. The optional aggregate of the magnetic nanoparticles is suitably porous and more preferably has dimensions that allow separation in a centrifuge 60. When using an alternative catalyst, such catalyst is again preferably chosen with a size allowing the separation thereof in the centrifuge 60. The polymer material is preferably loaded into the reactor vessel 10 in a ratio to the solvent in the range of 10:1 to 1:10. The order of adding the components (catalyst, waste material and solvent) is not relevant. It appears beneficial however to add the catalyst as a dispersion in the solvent such that it is added to the vessel 10 together with the solvent through the second inlet 12. Furthermore, the solvent may be pre-heated. The reactor system 100 could alternatively be elaborated as a batch system.

As shown in FIG. 1, the reactor vessel 10 is provided with a first outlet 21 configured for removal of a depolymerized mixture 31. The partly depolymerized mixture comprises monomers and light oligomers, as well as heavy oligomers, and may also comprise unreacted polymer, as well as other materials, as defined above. The partly depolymerized mixture 31 is led through a first filter unit 20, for instance comprising a strainer. The first filter unit 20 separates solid particles, such as metal, glass, stone and the like, and/or unreacted polymer from the depolymerized mixture 31. The thus cleaned partly depolymerized mixture exits the first filter unit as stream 32a. The partly depolymerized mixture stream 32 is predominantly liquid and is transferred by means of pump 41 to a heat exchanger 30 in which the partly depolymerized mixture stream 32 is cooled. The resulting cooled partly depolymerized mixture stream 32b is then fed through a second filter unit 40 and further into a downstream vessel 50, which may be provided with an inlet 51 for the addition of water or an aqueous solution. The second filter unit 40 may also be embodied as a strainer and serves to optionally separate polyolefins and other polymers from the partly depolymerized mixture stream 32b. The separated materials leave the second filter unit 40 as a stream 33. As shown in FIG. 1, another vessel 35 may be provided upstream of the second filter unit 40 in certain embodiments. The other vessel 35 may optionally be provided with an inlet 36 for the addition of water or an aqueous solution. The other vessel 35 helps in separating the polyolefins and other polymers from the partly depolymerized mixture stream 32b in second filter unit 40.

The downstream vessel 50 may in an embodiment be provided with mixing means to ensure adequate mixing of the cooled partly depolymerized mixture 32 and the optionally provided water or aqueous solution. Typically, such mixing means include a mixing chamber and a stirrer in whatever form. However, a stirrer may not be strictly necessary in dependence on the flow regime of the cooled partly depolymerized mixture stream 32b. Indeed, the cooled depolymerized mixture stream 32b may be supplied as a turbulent stream, and a mixing chamber without stirrer may then be sufficient. The mixing chamber is preferably part of the downstream vessel but may alternatively be implemented as a chamber upstream of the downstream vessel 50. The same remarks may be made with respect to the optional other vessel 35.

The optional water or aqueous solution added to the vessel 50 (and optionally to the other vessel 35) may act as coolant. It may be provided at ambient temperature or any higher temperature and is preferably liquid. Still, it is not excluded that separate cooling means are provided, and/or that the resulting stream would pass another heat exchanger downstream of the vessel 50 or other vessel 35. It is also possible to provide a temperature sensor in the downstream vessel 50 that is coupled to a controller configured to control the heat exchanger 30, in order to assure a sufficiently low temperature. Due to the cooling or optional addition of water or an aqueous solution, two phases may appear, of which the first is an aqueous phase comprising solvent, monomer and at least the light oligomers, as defined in the present invention. The second phase is a slurry comprising solvent, catalyst and heavy oligomers, as defined in the present invention. The phases exit the vessel 50 as stream 32c and the phases are then separated in a centrifuge separator 60, resulting in a first separated phase 61 that is further processed to obtain a depolymerized product, such as BHET, and a second separated phase 62 that is recycled. In FIG. 1, the second phase 62 is shown to be directly recycled to the reactor vessel 10 via a solvent buffer vessel 70. According to the invention, the second phase 62 comprises alcoholic solvent, more preferably ethylene glycol, water, heavy oligomers, optionally colorants, and (heterogeneous) catalyst. It may also comprise minor amounts of light oligomers and monomer. The inventors have found out that by providing heavy oligomers in the depolymerized mixture stream 32c and in the centrifuge 60, a much larger amount of the (heterogeneous) catalyst is separated into the second phase 62 then is possible according to the state of the art. The amount of (heterogeneous) catalyst that is recycled to the reactor vessel 10 is then also increased. As a result, a much lesser amount of the (heterogeneous) catalyst is separated into the first phase 61 and lost.

In one implementation, the recycling line though which the second phase 62 is led to the first solvent buffer vessel 70 comprises a distillation step so as to reduce the water content of the second phase 62. Preferably, the second phase 62 is finally fed back into the reactor vessel 10 with a water content of less than 10 wt. %, more preferably less than 5 wt. % or less than 2 wt. %., or even less than 1%.

The further processing of the first phase 61 comprises for instance a treatment with active carbon and one or more crystallisation treatments, so as to arrive at crystalline material of a raw material suitable for polymerization. Most preferably, the raw material is BHET, but it is further feasible to collect crystalline light oligomers, such as dimers, trimers and tetramers.

Although not shown in FIG. 1, the lines connecting the different units may be provided with valves for controlling the flow of the different mixture streams. It will be understood that such valves are under control of a controller, which is not shown either.

As further shown in FIG. 1, a stream 33 of solid particles and unreacted polymer is led to a second reactor vessel 80. The second reactor vessel 80 is arranged downstream of the first outlet 21 of the first reactor vessel 10. As such, the object of the second reactor vessel 80 is to achieve further depolymerization of the unreacted polymer that enters the second reactor vessel 80 through an inlet 81 thereof. The conditions in the second reactor vessel 80 are preferably maintained such that a substantially full conversion of the polymer into monomers is achieved in the second reactor vessel 80. This is in contrast with the conditions imposed in the first reactor vessel 10, which, according to an embodiment are such that depolymerization in the first reactor vessel 10 is performed merely partially to a non-full conversion of the polymer, so that heavy oligomers and eventually even polymer are still present when leaving the first reactor vessel 10 as stream 31. It has been found in experiments leading to this embodiment of the invention, that the presence of heavy oligomers is advantageous for the separation in the centrifuge 60 of the homogeneous or heterogeneous catalyst, even without adding water or another aqueous solution as a phase separation additive in the mixing vessel 50. In a preferred embodiment therefor, the vessel 50 does not include an inlet 51 for water or another aqueous solution.

As further shown in FIG. 1, the second reactor vessel 80 is provided with an outlet 82 configured for removal of a substantially fully depolymerized mixture 52. The depolymerized mixture comprises monomers (and maybe some light oligomers) and may also comprise other materials, as defined above. The fully depolymerized mixture 52 is led through a third filter unit 90, for instance comprising a strainer. The third filter unit 90 separates remaining solid particles, such as metal, glass, stone and the like, from the depolymerized mixture 52, and leads them away as stream 53. The thus cleaned depolymerized mixture exits the third filter unit 90 as stream 54. The substantially fully depolymerized mixture stream 54 is predominantly liquid and transferred to the heat exchanger 30 in which the substantially fully depolymerized mixture stream 54 is combined with the partly depolymerized mixture stream 32a and cooled. The resulting cooled depolymerized mixture stream 32b is then fed through the second filter unit 40 and further into a downstream vessel 50, as has been described above.

At least one further inlet 83 may be provided to the second reactor vessel 80. This inlet 83 may for instance be configured for optionally adding solvent and/or catalyst, or even more polymer waste material, to the second reactor vessel 80.

The second reactor vessel 80 may be provided with yet another inlet 84 that connects to an upstream second solvent buffer vessel 71. The second solvent buffer vessel 71 connects through line 72 to the first solvent buffer vessel 70 that is provided upstream and receives recycled solvent and (heterogeneous) catalyst through line 63. Optionally, the first solvent buffer vessel 70 is also provide with recycled solvent though line 64, or with fresh solvent and catalyst through line 65.

In one implementation of the reaction system and the use thereof for depolymerization, the second reactor vessel 80 is arranged as a batch reactor. This is the preferred choice for ensuring that substantially all the unreacted polymer coming from the first reaction vessel 10 will be depolymerized. The depolymerization temperature in the second reaction vessel 90 may for this purpose be maintained in the range of 170-250° C. for instance.

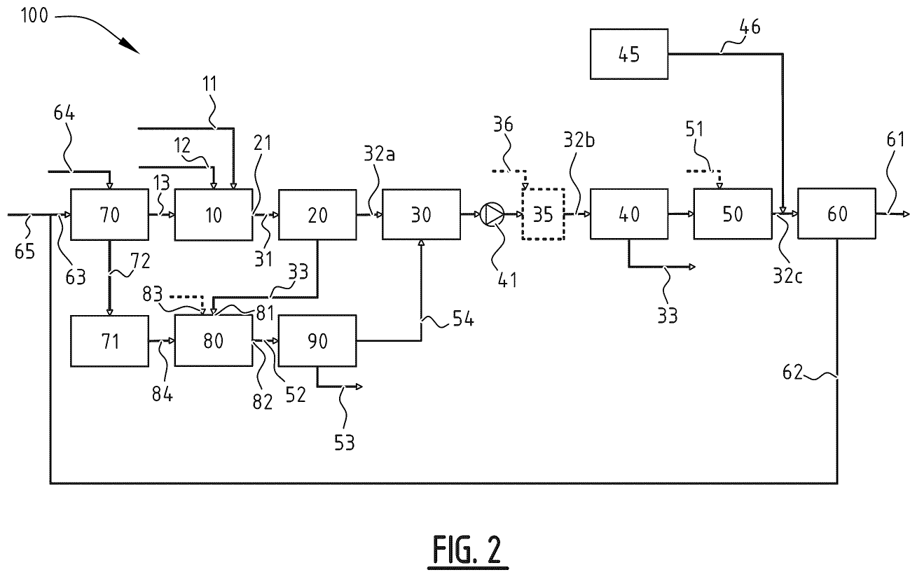

A second embodiment of the invention is shown in FIG. 2 as reactor system 101. In this embodiment, a separate source 45 of heavy oligomers is provided, as well as a line or conduit 46 that connects the heavy oligomer source 45 with one of the streams 32b and/or 32c. The conduit 46 may connect to the depolymerized product stream at position 32b between the heat exchanger 30 and the second filter unit 40, or, alternatively, between the second filter unit 40 and the vessel 50, or, alternatively just upstream from the centrifuge 60 and downstream from the vessel 50. The latter embodiment is shown in FIG. 2. The source 45 of heavy oligomers may comprise heavy oligomers that have been synthetized, or, alternatively, have been obtained by partly depolymerization of the primary polymer, or of another polymer.

In this embodiment, the conditions in the first reactor vessel 10 may be chosen such as to only partly depolymerize the primary polymer, as described above. Further polymerization is then carried out in the second reactor vessel 80. Additional heavy oligomers are then provided to the centrifuge 60 through the line 46. In another embodiment, it may also be possible to select the reaction conditions such that the primary polymer is substantially fully depolymerized in the first reactor vessel 10 to a full conversion. In that case substantially all the heavy oligomers needed to achieve the desired high recovery level of the (heterogeneous) catalyst are provided to the centrifuge 60 through the line 46. In this embodiment, it may even be possible to shut down the second reactor vessel 80. The solid particles exiting the first reactor vessel 10 as stream 33 may then be stored elsewhere.

The present invention will now be illustrated more in detail by reference to the following examples, which are not meant to limit the invention but are merely given as exemplary embodiments of the invention.

### EXAMPLES

Tests have been performed on coloured PET and on non-coloured PET as well. The results thereof are in the same order of magnitude for both conversion and selectivity towards BHET. As a consequence inventors conclude that a colour additive has hardly any or no impact in this respect. Even further, additives, such as pigments, can be removed from the degradation products, with ease.

The sourced raw (PET) material may comprise polyester clothing, PET carpet, PET material originating from the automotive industry, recycled PET, and multi-layered PET trays containing other polymers, such as PE and PP. The results thereof again are in the same order of magnitude with respect to selectivity and conversion.

The average residence time of the polymer in the reactor during which degrading is performed may be chosen from 30 sec.-5 hours, preferably from 60 sec.-2 hours, more preferably from 2-60 min, such as 5-30 min. Depending on e.g. reactor size and boundary conditions, longer or shorter periods may be used. For instance, a high pressure (500-3000 kPa) process at a temperature of between 150° C.-350° C. may lead to very short degrading times, in the order of minutes.

The reactor is selected from a (semi) continuous type, such as a continuous stirred tank reactor (CSTR), and a tube-like reactor, such as a loop reactor, a plug flow reactor, an oscillatory flow reactor, an N-unit loop reactor system, and a batch type, and combinations thereof.

In an example of the present method the degrading is performed at a temperature of 50° C.-500° C., preferably 90° C.-350° C., more preferably 150° C.-250° C., even more preferably 170° C.-220° C., such as 180° C.-210° C., e.g. 185° C. and 200° C. The preferred range is considered to relate to a relative mild temperature, especially as compared to prior art processes which are performed at temperatures above 300° C.

In an example of the present method the pressure is from 90 kPa-10.000 kPa, preferably 100 kPa-8.000 kPa, more preferably 200 kPa-2.000 kPa. Mild pressures in an example are an advantage over some prior art processes, which need to be performed at relatively high pressures, of e.g. 1000 kPa, often in combination with a high temperature. In a selection of a combination of temperature and pressure, a range of [T,P] from [180° C., 60 kPa] to [450° C., 8.200 kPa] may be chosen.

In an example of the present method the amount of catalyst is 0.001-35 wt. %, preferably 0.005-20 wt. %, more preferably 0.01-10 wt. %, even more preferably 0.05-0.15 wt. %, relative to a total weight of polymer provided. If the amount of catalyst is higher, a shorter reaction time may be obtained, whereas at a lower amount, longer reaction times are generally needed. Depending on further boundary conditions, one may vary the amount of catalyst.

In the case of depolymerizing PET, the catalyst may for instance be used in a ratio (weight to weight) of catalyst:PET from 1:5 to 1:2500, such as 1:1000-1:1500. In addition the amount of e.g. ethylene glycol:PET may vary from 1:2 to 1:20, such as 1:3 to 1:8. The waste polymers may relate to a single type of polymer, such as PET, PEF, PA, etc., and also to a mixture thereof. It typically comprises 50-99.9 wt. % of a specific polymer, such as PET, the remainder being impurities, other polymers, other compounds, etc.

### Comparative Experiment A: Full Conversion (FC)

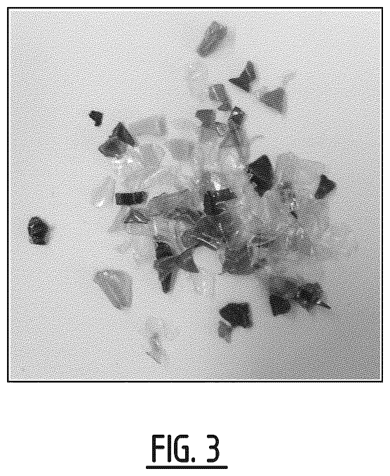

Depolymerization experiments were carried out using a 500 ml flask. An amount of 0.027 g of an ABC-complex catalyst comprising a catalyst entity C, an iron containing nanoparticle A, and a bridging moiety B connecting the catalyst entity C to said magnetic iron nanoparticle A, was used in combination with 33.4 g of post-consumed mixed-colored polyethylene terephthalate (PET) flakes (pieces of around 10×10 mm) and 250 g of ethylene glycol (EG). The weight ratio of catalyst complex to PET was therefore 1:1250 or 0.08 wt. %. FIG. 3 shows a photograph of the polyethylene terephthalate (PET) flakes used.

The round bottom flask was placed in a heating setup. The heating was started, and after 20 minutes, the reaction mixture had reached the reaction temperature of 197° C. After 300 min at 197° C., the reaction was stopped by cooling down below 160° C.

The reaction mixture was transferred to a centrifuge flask through a sieve filter to remove remaining solids. An amount of 1.3 g of remained solids was collected (non-PET materials, e.g., polyethylene, polypropylene) presented in the feedstock.

Water was added to the centrifuge bottle to obtain a water:EG ratio of 1:1. The mixture was centrifuged at 4000 rpm for 3 min. The supernatant was separated by decanting and let cooled down to crystallize the BHET product. BHET was filtered out from mother liquor using a Büchner filter and dried in a vacuum oven at 60° C.

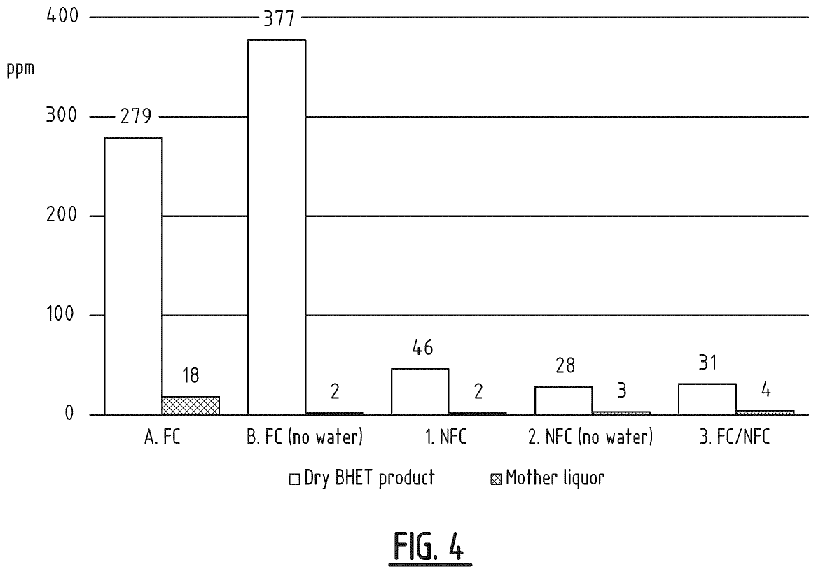

Dry BHET and mother liquor were analyzed by XRF to estimate separability. As shown in FIG. 4 under A. FC, amounts of 279 ppm and 18 ppm of iron were detected in dry BHET and mother liquor, respectively. The amount of dry BHET was 36.4 g. A separation efficiency of 39% was calculated.

### Comparative Experiment B: Full Conversion without the Addition of Water (FC(No Water))

The same procedure of depolymerization reaction described in Comparative Experiment A was used.

After 300 min at 197° C., the reaction was stopped by cooling down below 160° C. The reaction mixture was transferred to a centrifuge flask through a sieve filter to remove remained solids. 0.5 g of remained solids were collected (non-PET materials, e.g., polyethylene, polypropylene) presented in the feedstock.

The mixture was centrifuged at 4000 rpm for 3 min. The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 4 under B. FC (no water), amounts of 377 ppm and 2 ppm of iron were detected in dry BHET and mother liquor, respectively. The amount of dry BHET was 35.2 g. A separation efficiency of 22% was calculated.

### Example 1: Non-Full Conversion (NFC)

The same procedure of depolymerization reaction described in Comparative Experiment A was used.

After 120 min at 197° C., the reaction was stopped by cooling down below 160° C. The reaction mixture was transferred to a centrifuge flask through a sieve filter to remove remained solids. 16.5 g of remained solids were collected, consisting of unreacted PET and non-PET materials (e.g., polyethylene, polypropylene) presented in the feedstock.

Water was added to the centrifuge bottle to obtain the water:EG ratio of 1:1. The mixture was centrifuged at 4000 rpm for 3 min. A clear layer of oligomers (containing catalyst) was observed. The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 4 under 1. NFC, 46 ppm and 2 ppm of iron were detected in dry BHET and mother liquor, respectively. The amount of dry BHET was 17.2 g. A separation efficiency of 90% was calculated.

### Example 2: Non-Full Conversion without the Addition of Water (NFC (No Water))

The same procedure of depolymerization reaction described in Comparative Experiment A was used.

After 120 min at 197° C., the reaction was stopped by cooling down below 160° C. The reaction mixture was transferred to a centrifuge flask through a sieve filter to remove remained solids. 12.3 g of remained solids were collected, consisting of unreacted PET and non-PET materials (e.g., polyethylene, polypropylene) presented in the feedstock.

The mixture was centrifuged at 4000 rpm for 3 min. A clear layer of oligomers (containing catalyst) was observed. The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 4 under 2. NFC (no water), 28 ppm and 3 ppm of iron were detected in dry BHET and mother liquor, respectively. The amount of dry BHET was 21 g. The calculated separation efficiency was 89%.

### Example 3: Full Conversion+Non-Full Conversion (FC/NFC)

The same procedure of depolymerization reaction described in Comparative Experiment A was used.

After 300 min at 197° C., the reaction was stopped by cooling down to room temperature. 33.4 g of fresh post-consumed mixed colored polyethylene terephthalate (PET) flakes (pieces of around 10×10 mm) were added to the reaction mixture. The heating was started, and after 20 minutes, the reaction mixture had reached the reaction temperature of 197° C.

After 120 min at 197° C., the reaction was stopped by cooling down below 160° C. 14.1 g of remained solids were collected, consisting of unreacted PET and non-PET materials (e.g., polyethylene, polypropylene) presented in the feedstock.

Water was added to the centrifuge bottle to obtain the water:EG ratio of 1:1. The mixture was centrifuged at 4000 rpm for 3 min. A clear layer of oligomers (containing catalyst) was observed. The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 4 under 3. FC/NFC, 31 ppm and 4 ppm of iron were detected in dry BHET and mother liquor, respectively. The amount of dry BHET was 50.95 g.

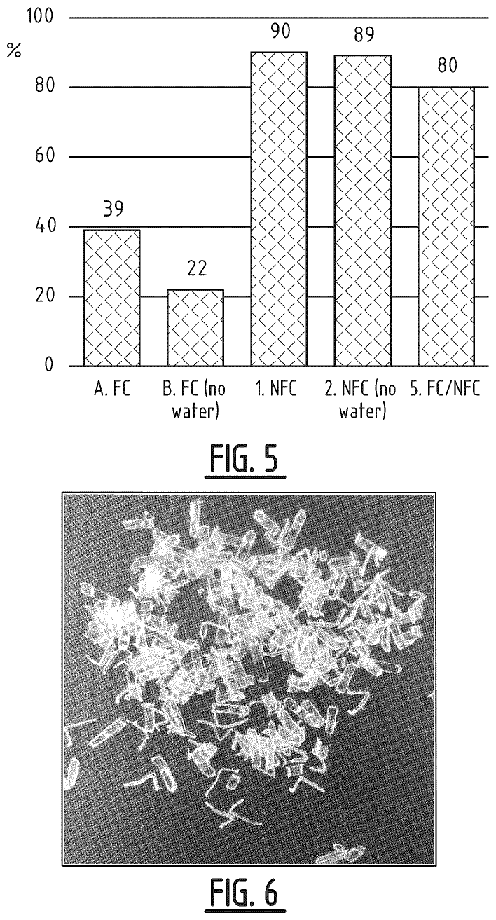

The results are summarized in FIGS. 4 and 5. FIG. 4 shows the concentration of Fe (in ppm) found in the recovered BHET and the mother liquor. FIG. 5 shows the separation efficiency in percentage (%). The results demonstrate that in a method according to the invention, the iron-based catalyst is recovered to a substantial degree, i.e. at least 10 times as best as in prior art methods. Indeed, only 28-46 ppm of catalyst remains in the dry BHET obtained, and 2-4 ppm in the mother liquor, while 279-377 ppm of catalyst is lost in the dry BHET according to known methods. The separation efficiency of the catalyst is 80-90%, while it is only 22-39% for the prior art method.

### Comparative Experiment C: Transparent Colorless PET. Full Conversion (FC)

Depolymerization experiments were carried out using a 500 ml flask. An amount of 0.027 g of ABC-complex catalyst was used in combination with 33.4 g of transparent polyethylene terephthalate (PET) flakes (pieces of 10×2 mm) and 250 g of ethylene glycol (EG). FIG. 6 shows a photograph of the transparent polyethylene terephthalate (PET) flakes used.

The round bottom flask was placed in the heating setup. The heating was started, and after 20 minutes, the reaction mixture had reached the reaction temperature of 197° C. After 300 min at 197° C., the reaction was stopped by cooling down below 160° C. The reaction mixture was transferred to a centrifuge flask through a sieve filter to remove remained solids. No remained solids were collected, indicating full-conversion of PET feedstock.

Water was added to the centrifuge bottle to obtain the water:EG ratio of 1:1. The mixture was centrifuged at 4000 rpm for 3 min. The supernatant was separated by decanting and let cooled down to crystallize the BHET product. BHET was filtered out from mother liquor using a Büchner filter and dried in a vacuum oven at 600° C.

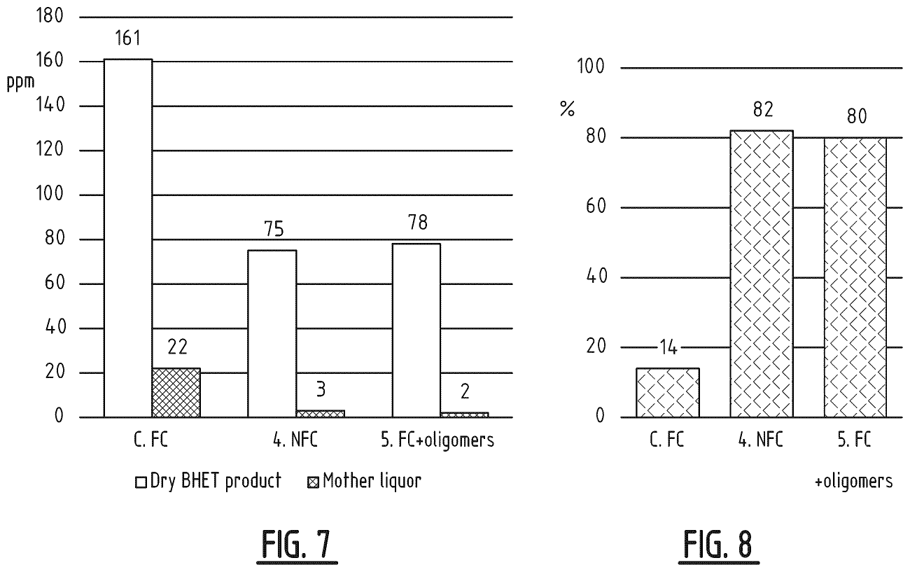

Dry BHET and mother liquor were analyzed by XRF to estimate separability. As shown in FIG. 7 under C. FC, 161 ppm and 22 ppm of iron were detected in dry BHET and mother liquor, respectively. The amount of dry BHET was 37.45 g. The calculated separation efficiency was 14%.

### Example 4: Transparent Colorless PET: Non-Full Conversion (NFC)

The same procedure of depolymerization reaction described in Comparative Experiment C was used.

After 180 min at 197° C., the reaction was stopped by cooling down below 160° C. The reaction mixture was transferred to a centrifuge flask through a sieve filter to remove remained solids. An amount of 8.36 g of remained solids was collected, consisting of unreacted PET.

Water was added to the centrifuge bottle to obtain the water:EG ratio of 1:1. The mixture was centrifuged at 4000 rpm for 3 min. A clear layer of oligomers (containing catalyst) was observed.

The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 7 under 4. NFC, 75 ppm and 3.2 ppm of iron were detected in dry BHET and mother liquor, respectively. The amount of dry BHET was 25.3 g. A separation efficiency of 82% was calculated.

### Example 5: Transparent Colorless PET: Non Full Conversion and Addition of Pre-Made Oligomers (NFC)

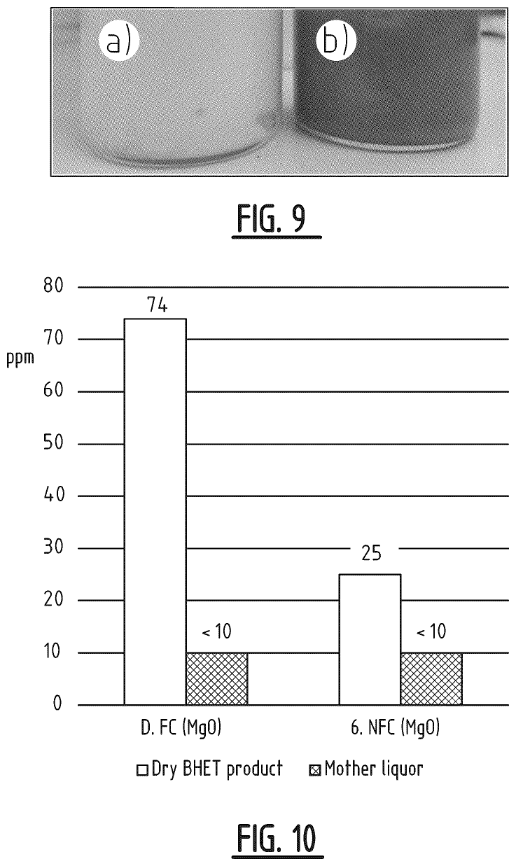

Oligomers were made in a non-full conversion NFC-depolymerization reaction. The experiments were carried out using a 500 ml flask. An amount of 0.014 g of Zn (II) Acetate catalyst was used in combination with 33.4 g of transparent polyethylene terephthalate (PET) flakes (pieces of 10×2 mm) and 250 g of ethylene glycol (EG). The round bottom flask was placed in the heating setup. The heating was started, and after 20 minutes, the reaction mixture had reached the reaction temperature of 197° C. After 120 min at 197° C., the reaction was stopped by cooling down below 160° C. The reaction mixture was transferred to a centrifuge flask through a sieve filter to remove remained solids. 10.9 g of remained solids were collected, consisting of unreacted PET. Water was added to the centrifuge bottle to obtain the water:EG ratio of 1:1. The mixture was centrifuged at 4000 rpm for 3 min. The white precipitate layer consisting of (heavy) oligomers with EG and water was collected at the bottom of the centrifuge jar (28.6 g). FIG. 9a shows a photograph of the centrifuge precipitate comprising the pre-made oligomers.

The same procedure of depolymerization reaction described in Comparative Experiment C was used. After 300 min at 197° C., the reaction was stopped by cooling down to room temperature.

An amount of 28.6 g of the white precipitate obtained from the NFC reaction with Zn (II) Acetate was added to the reaction mixture as heavy oligomers. The heating was started, and after 10 minutes, the reaction mixture had reached the temperature of 160° C. to dissolve the added oligomers. The reaction mixture was transferred to a centrifuge flask through a sieve filter to remove remained solids. No remained solids were collected, indicating full-conversion of PET feedstock and complete dissolution of the added oligomers.

Water was added to the centrifuge bottle to obtain the water:EG ratio of 1:1. The mixture was centrifuged at 4000 rpm for 3 min. A clear layer of oligomers (containing catalyst) was observed. The precipitate color has changed from white to brown, as shown in the photograph of FIG. 9.

Indeed, FIG. 9 shows a photograph of the centrifuge precipitate for the a) pre-made oligomers, and for the b) oligomers after full conversion (FC) depolymerization/separation. The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 7 under 5. FC+oligomers, 78 ppm and 2 ppm of iron were detected in dry BHET and mother liquor, respectively. The amount of dry BHET was 37.3 g. A separation efficiency of 80% was calculated.

The results are summarized in FIGS. 7 and 8. They show that in a method according to the invention, the iron-based catalyst is recovered to a substantial degree, i.e. better than in prior art methods. Indeed, only 75-78 ppm of catalyst remains in the dry BHET obtained, and 2-3 ppm in the mother liquor, while 161 ppm of catalyst is lost in the dry BHET according to known methods. The separation efficiency of the catalyst is 80-82%, while it is only 14% for the prior art method.

### Comparative Experiment D: Transparent Colorless PET: Full Conversion (FC)

Depolymerization experiments were carried out using a 500 ml flask. 0.034 g of MgO catalyst was used in combination with 33.4 g of transparent polyethylene terephthalate (PET) flakes (pieces of 10×2 mm) and 250 g of ethylene glycol (EG).

The round bottom flask was placed in the heating setup. The heating was started, and after 20 minutes, the reaction mixture had reached the reaction temperature of 197° C. After 300 min at 197° C., the reaction was stopped by cooling down below 160° C.

The reaction mixture was transferred to a centrifuge flask through a sieve filter to remove remained solids. No remained solids were collected, indicating full-conversion of PET feedstock. Water was added to the centrifuge bottle to obtain the water:EG ratio of 1:1. The mixture was centrifuged at 4000 rpm for 3 min. The supernatant was separated by decanting and let cooled down to crystallize the BHET product. BHET was filtered out from mother liquor using a Büchner filter and dried in a vacuum oven at 60° C.

Dry BHET and mother liquor were analyzed by XRF to estimate separability. As shown in FIG. 10 under D. FC (MgO), 74 ppm and <10 ppm of magnesium were detected in dry BHET and mother liquor, respectively. The amount of dry BHET was 34.1 g. The calculated separation efficiency was 64%.

### Example 6: Transparent Colorless PET: Non-Full Conversion (NFC)

The same procedure of depolymerization reaction described in Comparative experiment D was used.

After 120 min at 197° C., the reaction was stopped by cooling down below 160° C. The reaction mixture was transferred to a centrifuge flask through a sieve filter to remove remained solids. 5.8 g of remained solids were collected, consisting of unreacted PET.

Water was added to the centrifuge bottle to obtain the water:EG ratio of 1:1. The mixture was centrifuged at 4000 rpm for 3 min. The supernatant was separated by decanting and let cooled down to crystallize the BHET product. BHET was filtered out from mother liquor using a Buhner filter and dried in a vacuum oven at 60° C.

Dry BHET and mother liquor were analyzed by XRF to estimate separability. As shown in FIG. 10 under 6. NFC (MgO), 25 ppm and <10 ppm of magnesium were detected in dry BHET and mother liquor, respectively. The amount of dry BHET was 29.5 g. A separation efficiency of 74% was calculated.

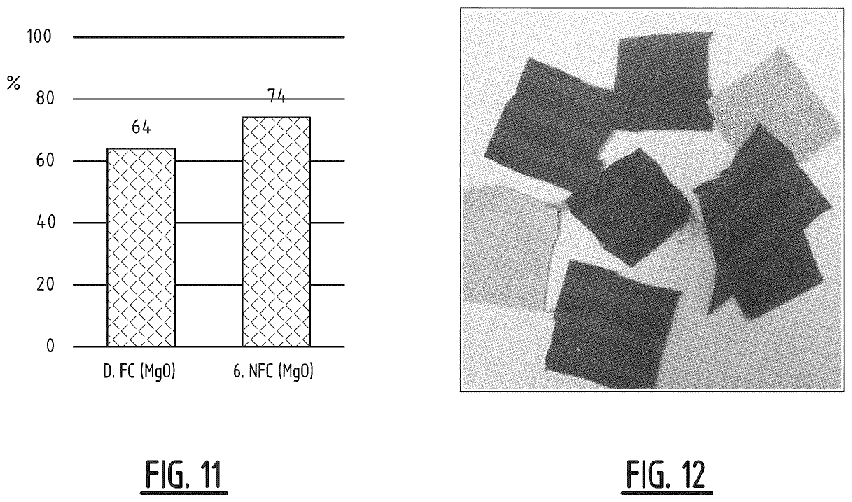

The results are summarized in FIGS. 10 and 11. They show that in a method according to the invention, the MgO catalyst is recovered to a substantial degree, i.e. better than in prior art methods. Indeed, only 25 ppm of catalyst remains in the dry BHET obtained, while 74 ppm of catalyst is lost in the dry BHET according to known methods. The amount of MgO catalyst in the mother liquor could not be measured since it was below measurement limits. The separation efficiency of the MgO catalyst is 74%, while it is 64% for the prior art method.

### Comparative Experiment E: Colored Textile PET: Full Conversion (FC)

Depolymerization experiments were carried out using a 500 ml flask. An amount of 0.034 g of ABC-complex catalyst was used in combination with 33.4 g of mixed colored polyethylene terephthalate (PET) textile (pieces of 2.5×2.5 cm) and 250 g of ethylene glycol (EG). FIG. 12 shows a photograph of the PET textile pieces used.

The round bottom flask was placed in the heating setup. The heating was started, and after 20 minutes, the reaction mixture had reached the reaction temperature of 197° C. After 180 min at 197° C., the reaction was stopped by cooling down below 160° C. The reaction mixture was transferred to a centrifuge flask through a sieve filter to remove remained solids. No remained solids were collected, indicating full-conversion of PET feedstock.

Water was added to the centrifuge bottle to obtain the water:EG ratio of 1:1. The mixture was centrifuged at 4000 rpm for 3 min. The supernatant was separated by decanting and let cooled down to crystallize the BHET product. BHET was filtered out from mother liquor using a Büchner filter and dried in a vacuum oven at 60° C.

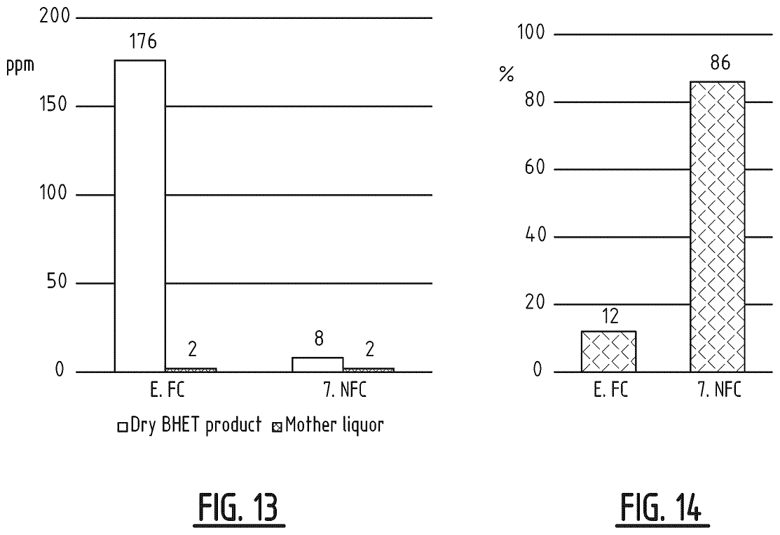

Dry BHET and mother liquor were analyzed by XRF to estimate separability. As shown in FIG. 13 under E. FC, 176 ppm and 2 ppm of iron were detected in dry BHET and mother liquor, respectively. The amount of dry BHET was 35.8 g. The calculated separation efficiency was 12%.

### Example 7: Colored Textile PET: Non-Full Conversion (NFC)

The same procedure of depolymerization reaction described in Comparative Experiment E was used.

After 55 min at 197° C., the reaction was stopped by cooling down below 160° C. Water was added to the centrifuge bottle to obtain the water: EG ratio of 1:1. The mixture was centrifuged at 4000 rpm for 3 min. A clear layer of unreacted PET fibers and oligomers (containing catalyst) was observed. The supernatant was separated by decanting and let cooled down to crystallize the BHET product. The precipitate was collected in a vial. BHET was filtered out from mother liquor using a Büchner filter and dried in a vacuum oven at 60° C.

Dry BHET and mother liquor were analyzed by XRF to estimate separability. As shown in FIG. 13 under 7. NFC, 8 ppm and 2 ppm of iron were detected in dry BHET and mother liquor, respectively. The amount of dry BHET was 11.7 g. A separation efficiency of 86% was calculated.

The results are summarized in FIGS. 13 and 14. They show that in a method according to the invention, the iron catalyst is recovered to a substantial degree, i.e. better than in prior art methods. Indeed, only 8 ppm of catalyst remains in the dry BHET obtained, while 176 ppm of catalyst is lost in the dry BHET according to known methods. The separation efficiency of the iron catalyst is 86%, while it is 12% for the prior art method.

1. A method for depolymerizing a polymer, the method comprising the steps of:
a) providing the polymer and a solvent in a reactor vessel to obtain a reaction mixture, the solvent being capable of reacting with the polymer to degrade the polymer into its monomers and oligomers;
b) providing a reusable catalyst in the reaction mixture being capable of catalysing said degradation;
c) degrading the polymer in the reaction mixture at degradation reaction conditions to obtain a depolymerized mixture comprising at least the monomers and light oligomers, having from 2 to 4 repeating units inclusively; and removing unreacted polymer, solid particles and very heavy oligomers having more than 200 repeating units from the depolymerized mixture after exiting the reactor,
d) recovering at least a part of the reusable catalyst from the depolymerized mixture;
e) recovering the monomers and the light oligomers from the depolymerized mixture;
wherein, during recovery of the reusable catalyst in step d), the depolymerized mixture comprises heavy oligomers having at least 5 repeating units and at most 200 repeating units, and wherein the removing of unreacted polymer and very heavy oligomers having more than 200 repeating units from the depolymerized mixture after exiting the reactor is carried out before step d).

2. Method as claimed in claim 1, wherein during recovery of the reusable catalyst in step d), the depolymerized mixture comprises heavy oligomers having at least 6 repeating units.

3. Method as claimed in claim 1, wherein during recovery of the reusable catalyst in step d), the depolymerized mixture comprises heavy oligomers having an upper bound of at most 100 repeating units.

4. Method as claimed in claim 1, wherein the heavy oligomers comprise repeating units of the polymer to be degraded.

5. Method as claimed in claim 4, wherein the heavy oligomers are formed by degrading the polymer in the reaction mixture at degradation reaction conditions during step c).

6. Method as claimed in claim 5, wherein the degradation reaction in step c) is stopped prematurely by removing the degradation reaction conditions.

7. Method as claimed in claim 5, wherein the degradation reaction temperature and/or time is reduced relative to the degradation reaction temperature and/or time needed for full degradation of said polymer into its monomers and light oligomers, comprising dimers, trimers and tetramers.

8. Method as claimed in claim 7, wherein the degradation reaction time is at most 0.95 times the degradation reaction time needed for full degradation of said polymer into said monomers and light oligomers.

9. Method as claimed in in claim 1, wherein said heavy oligomers also comprise oligomers having at least 5 repeating units of another polymer that differs from said polymer to be degraded.

10. Method as claimed in claim 9, wherein the other polymer comprises a condensation polymer that is degradable by said solvent at said degradation reaction conditions.

11. Method as claimed in claim 1, wherein said heavy oligomers are added to the depolymerized mixture after step c) and before or during step d).

12. Method as claimed in claim 1, wherein the amount of said heavy oligomers in the catalyst recovery step d) ranges from 0.1-50 wt. % relative to the total weight of the monomers and light oligomers in the depolymerized mixture.

13. Method as claimed in claim 1, wherein the amount of monomers and light oligomers in the depolymerized mixture ranges from 5-95 wt. % relative to the total weight of the monomers, light oligomers and heavy oligomers in the depolymerized mixture.

14. Method as claimed in claim 1, wherein unreacted polymer and very heavy oligomers with more than 100 repeating units are removed from the depolymerized mixture before step d).

15. Method as claimed in claim 14, wherein the removed unreacted polymer and optional very heavy oligomers are depolymerized to a substantially full conversion into monomers and light oligomers in a separate second reactor vessel by applying the steps a) to c) of claim 1.

16. Method as claimed in claim 15, wherein the fully depolymerized mixture that results from the depolymerization in the second reactor vessel is introduced in the depolymerized mixture before step d).

17. Method as claimed in claim 1, wherein other material present in the depolymerized mixture, such as polyolefins for instance, is at least partly removed from the depolymerized mixture after step c) and before step d).

18. Method as claimed in claim 1, wherein the catalyst recovery step d) comprises a phase forming step, comprising forming a first phase primarily containing the monomers and the light monomers and a second phase primarily containing said heavy oligomers and the catalyst, wherein the phase forming step comprises cooling the depolymerized mixture.

19. Method as claimed in claim 18, wherein the phase forming step is carried out substantially without adding water to the depolymerized mixture.

20. Method as claimed in claim 19, wherein the phase forming step is carried out using conditions such that said heavy oligomers at least partly precipitate from the reaction mixture.

21. Method as claimed in claim 13, further comprising separating said first phase primarily containing the monomers and light oligomers from said second phase primarily containing said heavy oligomers and the catalyst, wherein said separating is carried out at a temperature below 110° C.

22. Method as claimed in claim 17, wherein the light monomers recovery step e) comprises a step of crystallizing the monomers and light oligomers from said first phase primarily containing the light oligomers after the separating step.

23. Method as claimed in claim 1, wherein the step of providing the reusable catalyst comprises reusing the recovered catalyst.

24. Method as claimed in in claim 1, wherein the step of providing the reusable catalyst further comprises adding heavy oligomers from said another phase to the reaction mixture.

25. Method as claimed in in claim 1, wherein the solvent is a mono-alcohol or a di-alcohol.

26. Method as claimed in claim 1, wherein the polymer is a polycondensation polymer.

27. Method as claimed in claim, wherein the catalyst comprises a metal composition.

28. Method as claimed in claim 26, wherein the catalyst comprises a metal containing nanoparticle.

29. Method as claimed in claim 26, wherein the catalyst comprises a catalyst complex comprising a catalyst entity, said metal containing nanoparticle, and a bridging moiety connecting the catalyst entity to said magnetic nanoparticle.

30. A reactor system for recycling of waste material comprising a polymer suitable for depolymerization, the system comprising:
a first reactor vessel with at least one inlet for waste material and another inlet for providing a reusable catalyst to the first reactor vessel being capable of catalysing the depolymerization reaction of the polymer, and an outlet, which first reactor vessel is configured for depolymerizing the polymer into its monomers and oligomers; and which outlet is configured for exiting a depolymerized mixture;
a first filter unit arranged downstream of the outlet and configured for removing unreacted polymer, solid particles and very heavy oligomers having more than 200 repeating units from the depolymerized mixture after exiting the first reactor vessel, such that at least light oligomers having from 2 to 4 repeating units inclusively and heavy monomers having at least 5 and at most 200 repeating units remain present in the depolymerized mixture;
a heat exchanger provided downstream of the outlet and the first filter unit;
a separating unit provided downstream of the heat exchanger, the separating unit being configured for recovering at least a part of the reusable catalyst from the depolymerized mixture and/or for recovering the monomers and the light oligomers from the depolymerized mixture; and
a conduit system connecting the reactor system components, as well as pressure means for circulation purposes through the conduit system, wherein the conduit system comprises a feedback conduit for feeding the recovered part of the reusable catalyst back into the first reactor vessel.

31. The reactor system as claimed in claim 30, wherein the first filter unit is configured for removing very heavy oligomers with more than 100 repeating units from the depolymerized mixture after exiting the first reactor vessel.

32. The reactor system as claimed in claim 30, further comprising a separate second reactor vessel provided to receive the removed unreacted polymer and the optional very heavy oligomers from the first filter unit, wherein the second reactor is configured for depolymerizing the removed unreacted polymer and the optional very heavy oligomers to a substantially full conversion into monomers and light oligomers.

33. The reactor system as claimed in claim 32, wherein the second reactor vessel has an outlet configured for exiting the fully depolymerized mixture, and a conduit connecting the outlet with the heat exchanger.

34. The reactor system as claimed in claim 33, wherein the conduit connecting the outlet with the heat exchanger comprises a third filter unit arranged downstream of the outlet and configured for removing solid particles from the fully polymerized mixture.

35. The reactor system as claimed in claim 30, further comprising a second filter unit configured for removing other material present in the depolymerized mixture, such as polyolefins for instance, at least partly from the depolymerized mixture, wherein the second filter unit is provided downstream of the heat exchanger and upstream from the separating unit.

36. The reactor system as claimed in claim 30, further comprising a source of heavy oligomers other than the first reaction vessel, the source having an outlet configured for exiting the heavy oligomers from the source, and a conduit connected to the outlet and configured for adding the heavy oligomers to the depolymerized mixture in or downstream of the heat exchanger and/or in or upstream from the separating unit.

37. The reactor system as claimed in claim 30, wherein at least one solvent buffer vessel is arranged upstream of the first and/or second reactor vessel, an inlet of the at least one solvent buffer vessel being connected to the feedback conduit, and an outlet thereof being connected to the first reactor vessel and/or to the second reactor vessel.

38. The reactor system as claimed in claim 30, wherein the separating unit comprises a centrifuge, or a plurality of centrifuges provided in series, wherein any centrifuge may comprise a disc stack centrifuge.
